(19) United States
(12) Patent Application Publication
(10) Pub. No.: US 2009/0035275 A1
(43) Pub. Date: Feb. 5, 2009

(54) Methods and formulations for protecting cells, and for treating diseases and conditions by optimizing the intracellular concentration of NAD

(76) Inventors: John Geoffrey Pickering, London (CA); Eric Peter Van Der Veer, The Hague (NL)

Correspondence Address: McDERMOTT WILL & EMERY, 227 West Monroe Street, Chicago, IL, 60606-5096, US

(21) Appl. No.: 10/577,789

(22) PCT Filed: Oct. 29, 2004
(86) PCT No.: PCT/IB04/04472

§ 371 Date: Oct. 8, 2008

Related U.S. Application Data
(60) Provisional application No. 60/515,648, filed on Oct. 31, 2003.
(60) Provisional application No. 60/573,339, filed on May 24, 2004.
(60) Provisional application No. 60/624,625, filed on Oct. 25, 2004.

(51) Int. Cl. A61K 35/76; A61K 38/43; A61K 31/7088; A61K 31/665; A61P 9/00

(52) U.S. Cl. 424/93.2; 424/94.6; 514/44; 514/99

(57) ABSTRACT

Pharmaceutical and cosmetic formulations and methods for optimizing the intracellular concentrations of NAD are provided. The present methods and compounds relate to the use of PBEF, PRPP and various forms of nicotinamide, individually or in combination, for therapeutic, cyto-protective, cosmetic and anti-aging purposes. PBEF, PRPP and nicotinamide, individually or in combination, as administered according to the invention, increase the metabolic fitness, health and performance of the cell, and thereby increase the cell's level of health during its lifecycle. By way of the present formulations and methods, optimizing the intracellular concentration of NAD⁺ facilitates a balance among the numerous intracellular interactions of NAD⁺, and its related pathways, such that the health of the cell and its resistance to stress and trauma are increased. This increased robustness attendant to the invention also facilitates the delay of apoptosis.

### FIELD OF THE INVENTION

The present invention relates generally to the protective and therapeutic uses of substances which optimise the intracellular concentration and availability of nicotinamide adenine dinucleotide (NAD⁺), and to related methods. NAD⁺-optimising substances include those such as pre-B cell colony-enhancing factor (PBEF) and 5-phosphoribosyl-pyrophosphate (PRPP). The invention relates also to therapeutic and pharmaceutical formulations containing NAD-optimising compounds such as PBEF and PRPP, alone and in various combinations with other compounds such as nicotinamide.

### BACKGROUND OF THE INVENTION

Current techniques used to enhance cyto-protection or to inhibit aberrant cell functions tend to work by reducing environmental and metabolic factors that can harm or poison cells and tissues. Unfortunately, many of these techniques are limited to the dynamics of the scavenging of free radicals, and protective coatings.

Animal cells, such as human cells, experience stress many times throughout their lifecycles often causing injury, death or irreparable DNA damage. The source of the stress can be environmental, such as radiation, toxic substances, and physical factors experienced by the cell such as mechanical injury due to trauma, and exposure to extreme weather. Other stresses include those caused by sunlight, dehydration and exposure to caustic or otherwise harsh chemicals. Other sources of stress can occur during the natural phases of the cell cycle such as during times of proliferation and differentiation, and to the dynamics of carcinogenesis.

Revollo et al. postulate the regulatory interactions of Mampt, Nmnat and Sir2 on the intracellular dynamics of NAD. These interactions occur after the synthesis of NAD. The NAD Biosynthetic Pathway Mediated by Nicotinamide Phosphoribosy-ltransferase Regulates Sir2 Activity in Mammalian Cells, The Journal of Biological Chemistry, Sep. 20, 2004. INSERT re Amgen/Samal PBEF; Song et al PBEF Enhancing Factor; and Hasmann et al, FK86.

Until the present compounds and methods, the healing, pharmaceutical, cosmetic and metabolic arts have been lacking in effective methods and formulations to improve the metabolic fitness of cells. By improving cellular metabolic fitness, cells are best prepared to experience such commonly occurring stress without incurring damage that would prematurely shorten the life of the cell, cause the cell to function improperly or degrade the physical appearance of the cell.

This raised the possibility that PBEF was involved in the synthesis of NAD. NAD is well known for its role in regulating the redox state of the cell. However, recent work has identified a number of other important NAD-dependent reactions, including histone deacetylation. Unlike the redox system, these newly discovered reactions deplete the pool of cellular NAD, and sometimes contribute to harmful imbalances in the cell.

The present inventors have discovered that Optimizing the intracellular concentration of NAD⁺ facilitates a balance among the numerous intracellular interactions of NAD⁺ and its related pathways such that the health of the cell and its resistance to stress are increased. This increased robustness attendant to the invention also relates to a consequent delay of apoptosis of the cell.

### SUMMARY OF THE INVENTION

The present invention is based on the unexpected discovery that PBEF and PRPP, alone or in combination with one another, or in combination with one or more forms of nicotinamide, increase cell fitness, protect the cell against damage from stress factors, and increase the longevity of the cell.

Other aspects and features of the present invention will become apparent to those of ordinary skill in the art upon review of the following description of specific embodiments of the invention in conjunction with the accompanying Figures and claims. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The figures provided herein illustrate embodiments of the present invention, by way of example only, and not in a limiting way.

FIG. 1(A) shows Hoffman-modulated contrast images of HITB5 smooth muscle cells in M199 supplemented with 10% FBS (left images) and 6 days after culturing SMC's in serum-free M100 (right image;

FIG. 1(B) is a Northern Blot showing upregulation of the 3 major transcripts of PBEF in HITB5 SMC's following withdrawal of serum from cultures.

FIG. 1(C) is a Western Blot of cell lysates harvested from HITC6 SMC's before and after withdrawal of serum from cultures.

The images of FIG. 2 show the cellular elongation and aggregation of human Smooth Muscle Cells into multilayered ridges induced by the overexpression of PBEF. FIG. 2(A), shows Hoffman-modulated contrast images of sub-confluent (top panel) or post-confluent (middle panel) HITB5 Smooth Muscle Cells transduced with a retrovirus containing cDNA encoding EGFP alone (left image) or PBEF and EGFP from a bicistronic cassette (right image). The bottom panel of FIG. 2(A) depicts fluorescence images of the post-confluent SMC cultures, showing expression of the transgenes as indicated by EGFP fluorescence. Bar, 50 μm.

FIG. 2(B) shows the quantification of length-width ratios of control and PBEF-overexpressing Smooth Muscle Cells cultured in the presence of serum and 3 days after serum withdrawal.

FIG. 3 shows Western blots revealing expression of SMC differentiation markers in HITB5 SMC's infected with cDNA encoding EGFP alone (left image), and cDNA encoding both PBEF and EGFP (right image).

The images of FIG. 4 show the effect of PBEF of apoptosis. FIG. 4(A) shows cell accumulation over 11 days for control and PBEF-overexpressing HITB5 SMC's, cultured in M199 with 5% FBS.

FIG. 4(B) shows Thymidine incorporation into control and PBEF-overexpressing HITB5 SMC's, assessed by incubating cells in log-phase growth with 10 μCi/mL [³H]thymidine for 12 hours.

FIG. 4(C) presents fluorescence images of control and PBEF-overexpressing SMC's stained with Hoechst 33258 to identify nuclei (top panel), and for apoptotic nuclei by incubating with d-UTP fluorescein (bottom panel).

The images of FIG. 5 show the effect on SMC viability of the knockdown of PBEF expression and maturation induced by serum withdrawal.

FIG. 5(A) shows Hoffman-modulated images of control HITC6 SMC's and HITC6-siRNA SMC's. Western blots showing PBEF protein expression for each cell line are shown.

FIG. 5(B) shows the length-width ratios of 50 randomly selected cells expressing either nsRNA 1248 or siRNA 1248.

FIG. 5(C) is a Western blot showing reduced expression of h-Caldesmon in HITC6-siRNA 1248 Smooth Muscle Cells.

FIG. 6 is a phylogenetic tree showing a tight evolutionary relationship between bacterial nicotinamide phosphoribosyltransferases and eukaryotic PBEF, including human PBEF.

The depictions of FIG. 7 show the effects of increasing PBEF levels on the levels of NAD⁺. FIG. 7(A) shows the HPLC analyses of deproteinized nucleotide extracts obtained from HEK293 cells transfected with pQCXIP or pQCXIP-PBEF, and HITC6 SMC's transduced with pQCXIP or pQCXIP-PBEF.

FIG. 7(B) shows quantitative data from 3 separate experiments for each cell type of FIG. 7(A), including Western blot insets depicting representative PBEF expression for control and for cells overexpressing PBEF.

FIG. 8 shows that increasing the levels of PBEF increases NAD⁺-dependent histone deacetylase activity in human Smooth Muscle Cells.

The images of FIG. 9 show the effect of overexpression of PBEF on the phenotype, vessel chimerism and investment of Smooth Muscle Cells. FIGS. 9(A) through 9(D) show sections of matrigel implants loaded with human SMC's. FIGS. 9(E) through 9(I) show sections stained with h-caldesmon and h-calponin. FIG. 9(J) illustrates the quantification of the proportion of microvessels invested by at least one EGFP-positive SMC.

FIG. 10 is a schematic diagram of the structure of PPRP;

FIG. 11 is a diagram of the structure of the essential layers and components of human skin;

FIGS. 12 A-C are phase contrast photomicrographs at 20× of vector-transduced or PBEF-overexpressing HITC6 SMC's in response to treatment with 5-phosphoribosyl-pyrophosphate (PRPP), demonstrating that the PRPP increases the health of smooth muscle cells compared to control cells;

FIGS. 13 A-B are phase contrast photomicrographs at 20× magnification of vector-transduced HITC6 SMC's in response to treatment with 5-phosphoribosyl-pyrophosphate (PRPP), demonstrating that the PRPP increases the health of smooth muscle cells compared to control cells;

FIGS. 14 A-B are phase contrast photomicrographs at 10× magnification of vector-transduced HITC6 SMC's in response to treatment with 5-phosphoribosyl-pyrophosphate (PRPP). SMC's were cultured in M199 1% FBS for 24 hours prior to addition of 500 uM PRPP.

FIG. 15 shows the salient biosynthetic pathways involved in the synthesis of NAD.

FIG. 16 shows the salient biosynthetic pathways involved in the utilization and regeneration of NAD.

It is an object of the invention to provide methods for protecting cells and tissues from harm by optimising the levels or concentrations of one or more forms of NAD in the cells and tissues.

It is a similar object of the invention to provide methods for repairing and healing cells and tissues from harm by optimising the levels or concentrations of one or more forms of NAD in the cells and tissues.

It is a further object of the invention to provide pharmaceutical formulations efficacious in optimising the levels or concentrations of one or more forms of NAD in the cells and tissues.

It is a further object of the invention to provide cosmetic formulations efficacious in optimising the levels or concentrations of one or more forms of NAD in the cells and tissues.

### DETAILED DESCRIPTION

The term “a cell” as used herein includes a single cell as well as a plurality or population of cells. Administering an agent to a cell includes both in vitro and in vivo administrations.

The term “effective amount” as used herein means an amount effective, at dosages and for periods of time necessary to achieve the desired result.

The term “animal” as used herein includes all members of the animal kingdom, including humans.

Pharmaceutical Compositions may be prepared using standard techniques known in the art.

In one embodiment, there is provided a method for treating a disease state characterized by cells with a non-optimal NAD cycle. The patient may be any animal, including a mammal, including a human.

“Treating” a condition or disease state refers to an approach for obtaining beneficial or desired results, including clinical results. Beneficial or desired clinical results can include, but are not limited to, alleviation or amelioration of one or more symptoms or conditions, diminishment of extent of disease, stabilization of the state of disease, prevention of development of disease, prevention of spread of disease, delay or slowing of disease progression, delay or slowing of disease onset, amelioration or palliation of the disease state, and remission (whether partial or total), whether detectable or undetectable. “Treating” can also mean prolonging survival of a patient beyond that expected in the absence of treatment. “Treating” can also mean inhibiting the progression of an aberrant condition, slowing the progression of injury, aging or malfunction temporarily, although more preferably, it involves halting the progression of the same permanently. As will be understood by a skilled person, results may not be beneficial or desirable if, the treatment results in adverse effects on the patient treated that outweigh any benefits effected by the treatment.

Through the work of the present inventors, the importance of maintaining an optimal concentration of intracellular NAD+ to the robustness and completeness of cellular function is manifest in a number of ways, particularly when viewed in the context of the experiments reported herein. Moreover, the discovery of the present invention is unexpected, particularly in view of the related work of others.

In accordance with these and other objects, the invention provides formulations and methods for treating diseases or conditions in an animal. In one preferred embodiment, the method comprises the step of optimizing the intracellular concentration of PBEF in the cells of at least one target tissue of the animal, wherein the optimizing of the concentration of PBEF is effected by increasing the intracellular, or endogenous, concentration of the PBEF of the animal by a sufficient amount of PBEF.

In one preferred embodiment, optimization of the concentration of PBEF can be effected by administering to the animal a sufficient amount of PBEF to increase the intracellular concentration of the PBEF. The administration of the PBEF is preferably by at least one route, and the at least one route can be one or more of injection, oral administration, anal or other colonic administration, inhalation, intra-peritoneal administration, topical administration, intra-organ administration, infusion of a target tissue, transdermal and parenteral administration, including intravenous, intraperitoneal, subcutaneous, intramuscular, trans-epithelial, nasal, intrapulmonary, intrathecal, rectal and topical modes of administration or any other efficacious means. For example, in accordance with other objects of the invention, the optimization of PBEF levels can be performed by the methods of gene therapy, including the use of one or more viral vectors, such as adenoviruses, lentiviruses, adeno-associated viruses and non viral plasmid and cosmid vectors, and any other viral or prion vector amenable to optimizing the endogenous production and optimization of intracellular levels of PBEF.

Similarly, the present methods may be effected by promoting the endogenous production of PBEF in the cells of at least one target tissue of the animal, or in the whole animal, such as a human. Thus, promotion of intracellular production of PBEF can be effected, for example, by up-regulating the nucleic acid processes or mechanisms which support the production of PBEF, or by up-regulating the nucleic acid processes which increase the endogenous production of PBEF. Moreover, the present methods may be effected wherein the promotion of intracellular production of PBEF is effected by down-regulating the nucleic acid processes or mechanisms which repress the production of PBEF. Alternatively, the present methods may be practiced wherein the optimization of PBEF is effected by increasing the intracellular concentration of at least one modulator of PBEF, for example, by administering to the animal, such as a human, an effective amount of the modulator.

Administration of the modulator may be by any route known, and is preferably by at least one route, the at least one route being selected from routes such as injection, oral administration, anal or other colonic administration, inhalation, intra-peritoneal administration, topical administration, intra-organ administration, infusion of a target tissue, transdermal and parenteral administration, including intravenous, intraperitoneal, subcutaneous, intramuscular, trans-epithelial, nasal, intrapulmonary, intrathecal, rectal and topical modes of administration. In one preferred embodiment the modulator is PRPP.

In another preferred embodiment, the increase of PBEF is effected by promoting the endogenous production of PRPP in the cells of at least one target tissue of the animal, or in the whole animal, such as a human. The promotion of intracellular, or endogenous, production of PBEF may be effected, for example, by up-regulating the nucleic acid processes or mechanisms which increase the production of PRPP, or by down-regulating the nucleic acid processes or mechanisms which repress the production of PRPP.

In accordance with other methods of the invention, PRPP can be given in any efficacious form, or in combination with PBEF, or in combination with at least one form of nicotinamide, or in combination with PBEF and at least one form of nicotinamide. The nicotinamide may be in any efficacious form, such as in a substituted form, or in the form of one or more of nicotinic acid; nicotinic acid ribonucleotide; nicotinic acid ribonucleotide, reduced form; nicotinamide ribonucleotide; nicotinamide ribonucleotide, reduced form; nicotinic acid adenine dinucleotide; nicotinic acid adenine dinucleotide, reduced form; nicotinamide adenine dinucleotide (NAD); nicotinamide adenine dinucleotide phosphate (NADP); nicotinamide adenine dinucleotide, reduced form (NADH); and nicotinamide adenine dinucleotide phosphate, reduced form (NADPH) and pharmaceutically acceptable salts thereof.

The present methods and formulations may be used to treat any disease or condition, and particularly those involving the disruption, harm or imbalance of the NAD pathways of cells including those diseases and conditions involving the vascular system including the heart, blood vessels and other portions of the cardiovascular system. Examples of such diseases and conditions include vascular insufficiency, vascular weakness, progeria, premature senescence of one or more tissues, aging, severe stress on one or more tissues, atherosclerosis, arteriolesclerosis and re-vascularization of injured or weakened tissues or organs. The present methods and formulations may be used to treat any disease or condition which is a result of sever stress wherein the severe stress on one or more tissues is due to one or more of injury, malnutrition, disease, toxic shock and exposure.

Also in accordance with the present methods, the optimization of PBEF may be effected by increasing the intracellular concentration of at least one precursor of PBEF, for example, by administering to the animal an effective amount of the precursor.

Preferably, the administration of the precursor is by at least one route, and the at least one route is one or more of injection, oral administration, anal or other colonic administration, inhalation, intra-peritoneal administration, topical administration, intra-organ adminis-tration, infusion of a target tissue, transdermal and parenteral administration, including intravenous, intraperitoneal, subcutaneous, intramuscular, trans-epithelial, nasal, intra-pulmonary, intrathecal, rectal and topical modes of administration.

In one aspect, the precursor may be at least one form of nicotinamide.

In another aspect, the nicotinamide may be substituted or in the form of one or more of nicotinic acid; nicotinic acid ribonucleotide; nicotinic acid ribonucleotide, reduced form; nicotinamide ribonucleotide; nicotinamide ribonucleotide, reduced form; nicotinic acid adenine dinucleotide; nicotinic acid adenine dinucleotide, reduced form; nicotinamide adenine dinucleotide (NAD); nicotinamide adenine dinucleotide phosphate (NADP); nicotinamide adenine dinucleotide, reduced form (NADH); and nicotinamide adenine dinucleotide phosphate, reduced form (NADPH) and pharmaceutically acceptable salts thereof.

In accordance with still other advantages of the present invention, pharmaceutical and cosmetic formulations are provided. Formulations for optimizing the intracellular concentration of NAD of the invention include one or more of effective amounts of PRPP, PBEF, and nicotinamide. In compositions of the invention comprising nicotinamide, the nicotinamide may be substituted or in the form of one or more of nicotinic acid; nicotinic acid ribonucleotide; nicotinic acid ribonucleotide, reduced form; nicotinamide ribonucleotide; nicotinamide ribonucleotide, reduced form; nicotinic acid adenine dinucleotide; nicotinic acid adenine dinucleotide, reduced form; nicotinamide adenine dinucleotide (NAD); nicotinamide adenine dinucleotide phosphate (NADP); nicotinamide adenine dinucleotide, reduced form (NADH); and nicotinamide adenine dinucleotide phosphate, reduced form (NADPH) and pharmaceutically acceptable salts thereof.

A pharmaceutical or cosmetic composition of the invention may further comprise one or more of an effective amount of a pharmaceutically effective vehicle, a pharmaceutically effective diluent, a pharmaceutically effective cream, a pharmaceutically effective excipient, one or more pharmaceutically effective micelles, a pharmaceutically effective carrier, pharmaceutically acceptable concentrations of salt, buffering agents, preservatives and various compatible carriers. Preferably, compositions of the invention are adaptable for administration by at least one route, and the at least one route is one or more of injection, oral administration, anal or other colonic administration, inhalation, intra-peritoneal administration, topical administration, intra-organ administration, infusion of a target tissue, transdermal and parenteral administration, including intravenous, intraperitoneal, subcutaneous, intramuscular, trans-epithelial, nasal, intrapulmonary, intrathecal, rectal and topical modes of administration.

In accordance with still other objects of the invention, its compositions may be provided in the form of one or more of ingestible tablets, buccal tablets, troches, capsules, elixirs, suspensions, micelle encapsulations, syrups, wafers and the like, or enclosed or enclosable within hard or soft shell gelatin capsules. Moreover, the present compositions may further comprise one or more of an effective amount of a cosmetically effective vehicle, a cosmetically effective diluent, a cosmetically effective cream, a cosmetically effective excipient, one or more cosmetically effective micelles, a cosmetically effective carrier, cosmetically acceptable concentrations of salt, buffering agents, preservatives and various cosmetically compatible carriers.

As one of skill in the pharmaceutical or cosmetic arts will comprehend, numerous combinations and formulations of PBEF, PRPP and nicotinamide are within the scope and spirit of the invention, as are numerous variations of the present methods.

Vascular smooth muscle cells (SMC's) can exist in an immature state with the capacity to proliferate and migrate (1). The switch from a proliferative/migratory SMC to a contractile SMC is referred to as SMC maturation and is a process that is central to vascular development, stability, and physiologic function (2). Maturation of SMC's is necessary to stabilize newly formed blood vessels and confer vasomotor reactivity (3). Similarly, replicating SMC's in injured arteries must eventually mature to a quiescent phenotype to terminate the remodeling process.

The primary function of vascular SMC's in their quiescent state is to contract and provide vascular tone (Owens, 1995) (Sobue, 1998) (Rybalkin, 2003). The healthy SMC, which morphologically is similar to HITB5 or HITC6 SMC's that have been cultured in serum-free media for prolonged periods (more than 72 hours) is characterized by an elongated appearance, increased expression of SMC contractile proteins, such as h-caldesmon, metavinculin, smooth muscle myosin heavy chain and calponin and a marked decrease in apoptosis (Li, Circ. Res, 1999). We have found that HITB5 and HITC6 SMC's that are in this state have a significant increase in their transcript and protein levels of PBEF, demonstrating that PBEF is a novel factor involved in regulating SMC differentiation and maturation.

The molecular basis by which an immature SMC shifts to a functionally contractile cell is incompletely defined (4). This is partly due to a paucity of culture systems that recapitulate this critical, late phase of the SMC developmental program. Recently however, we cloned three adult vascular SMC lines that, in contrast to other human SMC preparations, could reversibly convert between a spread, immature state when cultured in the presence of serum to a highly elongated, mature state after serum withdrawal (5, 6). Whereas cultured human SMC's often die upon serum withdrawal, these cells displayed decreased apoptosis, increased contractile protein expression, and the ability to contract in response to vasoactive agonists. This system therefore provided us with an opportunity to seek out factors that enable an activated adult SMC to return to a metabolically quiescent cell specialized to contract. Accordingly, we undertook differential display PCR and high-density microarray analyses to identify genes that were differentially expressed as a homogenous population of human SMC's executed this key shift in phenotype.

These surveys identified pre-B cell colony-enhancing factor (PBEF) as being consistently upregulated as SMC's switched to the mature, quiescent state. PBEF is a 5255 kD protein that has been proposed to be a cytokine (7). Reported actions in this regard include synergizing with other cytokines to stimulate the maturation of pre-B cells (7), stimulating the expression inflammatory cytokines in amniotic epithelial cells (8), and prolonging neutrophil survival (9). However, the contention that PBEF is a secreted cytokine is controversial. PBEF does not have a signal sequence for secretion and the presence of PBEF in culture media has been suggested to be a consequence of activation-induced cell death, rather than secretion by either a classical or alternative pathway (10, 11). Moreover, PBEF has sequence similarity with bacterial nadV, a protein that confers bacteria with the ability to grow in nicotinamide adenine dinucleotide (NAD⁺)-deficient conditions (12, 13). In keeping with this, Rongvaux and co-workers have shown that mouse PBEF functions within the cell as a nicotinamide phosphoribosyltransferase (10). This enzyme catalyzes the rate-limiting step in the salvage pathway for NAD⁺ biosynthesis, whereby nicotinamide that is generated during NAD⁺-consuming reactions is utilized to regenerate NAD⁺ (14, 15).

The experimental data reported here indicates that phenotype switching of human vascular SMC's is dependent on PBEF. Whereas SMC's deficient in PBEF were compromised in their ability to elongate and express SMC differentiation markers, genetic augmentation of PBEF expression promoted SMC survival and conversion to a mature phenotype. These actions were associated with an increase in steady state NAD⁺ levels and increased NAD⁺-dependent histone deacetylase activity. SMC's with augmented expression of PBEF manifested enhanced ability to associate with endothelial cells and wrap around nascent blood vessels in a human-mouse chimeric vascular development model. These findings establish PBEF as a novel, intracellular regulator of vascular SMC phenotype and implicate PBEF-mediated NAD⁺ flux as a driver of human SMC maturation.

The mechanism by which PBEF exerts its pleiotropic effects is controversial. It has been suggested that PBEF functions as a secreted cytokine (Samal, 1994) (Jia, 2004), while it has also been reported to be involved in the salvage pathway of NAD+ biosynthesis as a nicotinamide phosphoribosyltransferase (Rongvaux, 2002) (Martin, 2001). From the results shoen herein, we believe that PBEF is an intracellular protein that catalyzes the rate-limiting reaction of the salvage pathway. This pathway involves the conversion of nicotinamide (NAm) and 5-phosphoribosyl-pyrophosphate (PRPP) to nicontinamide mononucleotide (NMN) (Pilz, JBC, 1984) (Willis, Adv. Enzyme Reg., 1989). In resting human lymphocytes, the PRPP pools are very low (Snyder, JCI, 1976), and these cells were characterized by an impaired ability to generate ATP, inability to respond to mitogenic stimuli, while activation of these cells results in expansion of NAD+ and PRPP pools concomitant with the repair of DNA single-strand breaks (Johnstone, Eur. J. Biochem, 1984) (Berger, Exp. Cell Res., 1982) (Williams, Exp. Cell Res., 1985). Carson, D. A., Seto, S., and Wasson, D. B. Pyridine Nucleotide Cycling and Poly(ADP-Ribose) Synthesis in Resting Human Lymphocytes. Journal of Immunology. 138: 1904-1907, 1987. Willis, R. C., Nord, L. D., Fujitaki, J. M., and Robins, R. K. Potent and Specific Inhibitors of Mammalian Phosphoribosylpyrophosphate (PRPP) Synthetase. Advances in Enzyme Regulation. 28: 167-180, 1989.

PPRP and Nicotinamide are molecules critical for the regeneration of NAD in animal cells. NAD+ is a critical coenzyme involved in oxidation-reduction reactions in the cell. Cellular maintenance of NAD+ levels for redox reactions is likely tightly regulated due to its critical importance in energy generation. However, recent work has identified a number of other NAD+-dependent reactions that, unlike the redox system, deplete the free pool of intracellular NAD+. As such, regeneration of NAD+ can be achieved by de novo biosynthesis in primarily the liver or via the salvage pathway in the peripheral tissues (Magni, CMLS, 2004) (Bender, Brit. Jour. Nutr, 1988). The catabolism of NAD(P)+ in the liver results in the release of nicotinamide into the blood, which is utilized by peripheral tissues for the regeneration of NAD+ (Bender, 1988). Conversion of nicotinamide to nicotinic acid by nicotinamide deamidase is an important aspect of NAD+ generation in bacteria and yeast (Anderson, Nature, 2003) (Schuette, Am. J. Physiol, 1983). However, it is believed that nicotinamide deamidase is not expressed in mammals (Schuette, Am. J. Physiol, 1983) (Rongvaux, Bioessays, 2003). Therefore, conversion of nicotinamide and 5-phosphoribosyl pyrophosphate to NMN by PBEF is one critical step for the regeneration of NAD+ in peripheral tissues (Rongvaux, Eur. J. Biochem, 2002), such as SMC's. The marked upregulation of PBEF in differentiating SMC's demonstrates that the differentiation of SMC's is an NAD+ consuming process.

The following experimental methods were employed to elucidate the data provided herein. Unnecessary details of the employed methods have been omitted for the sake of efficiency. Nonetheless, one of skill in the art will comprehend with certainty the significance and scope of the results reported herein.

Smooth Muscle Cell Culture lines were employed to assess the role of PBEF. Experiments were performed using the maturation-competent human vascular SMC lines, HITB5 and HITC6, generated from the human internal thoracic artery, as described previously (5, 6). SMCs were maintained in M199 (GibcoBRL, Burlington, ON) supplemented with the designated concentration of FBS (Hyclone). HEK293 cells were grown in DMEM with 10% FBS.

Overexpression of PBEF in Human Smooth Muscle Cells was evaluated by use of a viral vector. A retroviral gene delivery system was used to generate human SMCs stably overexpressing PBEF. Full-length cDNA encoding PBEF was amplified from HITB5 SMC mRNA by RT-PCR and subcloned into the pIRES-EGFP vector (Clontech). The PBEF-IRES-EGFP bicistronic fragment was then excised using XhoI and NotI and inserted into the retroviral expression vector pLNCX2 (Clontech). A second retroviral expression construct was generated by inserting PBEF cDNA into pQCXIP-IRES-PURO (Clontech). Retrovirus containing the cDNA of interest was obtained by calcium phosphate-mediated transfection of the Phoenix-amphotropic retrovirus packaging cell line (kindly provided by Dr. G Nolan, Stanford University Medical School, CA, distributed by ATCC, Manassas, Va.) as described previously (29). Virus-containing supernatant was added to proliferating SMCs and stable transductants were selected with 500 μg/ml G418 for 14 days, for pLNCX2-based constructs, and with 3 μg/ml puromycin for 48 hours, for pQCXIP-based constructs. Overexpression of PBEF was confirmed before each experiment by Western blot analysis.

Western Blot Analyses were employed to assay marker expression. Expression of PBEF and SMC differentiation markers was assessed by Western blot analysis with chemiluminescence detection, as described (29). Equal amounts of protein were resolved on 12% (for PBEF and α-tubulin), 9% (for smoothelin A and smoothelin B) and 6% (for caldesmon and vinculin-metavinculin) SDS-polyacrylamide gels and transferred PVDF membranes (Immobilon, Millipore). PBEF was detected using a polyclonal rabbit antibody against human PBEF (1857, 1:5000, kindly provided by Amgen). Monoclonal antibodies were used to detect heavy (h)-caldesmon (clone hHCD, 1:1000, Sigma), smoothelin isoforms A and B (clone MAB3242, 1:500, Chemicon), vinculin-metavinculin (clone VIN-11-5, 1:2000, Sigma), and α-tubulin (clone B-5-1-2; 1:16000, Sigma).

Cell Proliferation, DNA Synthesis, and Apoptosis were also evaluated. To assess SMC proliferation, cells were plated at a density of 3000 cells per cm² and cultured in M199 containing 5% FBS. Triplicate wells were harvested at the designated times and counted using a hemacytometer. To quantify DNA synthesis, cells in log-phase growth were incubated for 12 hours with [³H]thymidine (10 μCi/mL) and TCA-precipitable counts determined as described (5). Thymidine incorporation was expressed relative to DNA content, quantified by spectrofluorimetry of an aliquot of cell lysate incubated with 500 μg/ml Hoechst 33258. Apoptosis was assessed in SMCs seeded on glass coverslips by in situ end-labeling of DNA fragments using terminal deoxynucleotide transferase and fluorescein 12-dUTP (Promega) (5). Cells were fixed with 4% paraformaldehyde and counterstained with Hoechst 33258.

Knockdown of PBEF by RNA Interference was evaluated with a viral vector. PBEF knockdown was accomplished by infecting human SMC's with retrovirus containing sequences encoding hairpin siRNA fragments. Complementary oligodeoxynucleotides were synthesized, annealed, and inserted between the BamHI and EcoRI sites of the retroviral expression vector pSIREN-RetroQ. Three different targeting sequences were used, each consisting of 19 nucleotides starting at nucleotides 147, 384, and 1248 of the PBEF coding sequence (siRNA147 5′-GGAAGGTGAAATATGAGGA-3′; siRNA384 5′-ATGTTCTCTTCACGGTGGA-3′; siRNA1278 5′-AGGGCCGATTATCTTTACA-3′). Each sequence was separated by a 9-nucleotide noncomplementary spacer from the reverse complement of the same 19nucleotide sequence. Blast search confirmed that only the PBEF gene was targeted. Control inserts contained the gene-specific 19-nucleotide sequence and hairpin loop sequence but not the antisense component. Infection of SMCs was performed as described above and cells were selected using 3 μg/ml puromycin for 48 hours.

Real-time RT-PCR was used to analyze nucleic acid evidence. RNA was harvested using RNeasy mini-column and reagents (Qiagen) and subjected to DNaseI treatment. Probe (5′-CAGTTGCTGATCCCA-3′) and flanking primers (5′-primer 5′-TGCAGCTATGTTGTAACCAATGG-3′; 3′-primer 5′-ACAAAAGGTCGAAAAAGGGCC-3′) for Taqman real-time RT-PCR for PBEF were designed using Primer Express software (Applied Biosystems). Reactions were performed using an ABI-Prism 7900 Sequence Detection System (Applied Biosystems). Optimum signal was obtained with concentrations of 200 nM, 300 nM and 50 nM for the probe, 5′ primer, and 3′ primer, respectively. Standard curves were generated using RNA derived from human aortic SMCs, enabling correlation of the determined threshold cycle to transcript abundance. GAPDH transcript abundance was used as an endogenous RNA control (Assays on Demand Hs9999905, Applied Biosystems) to which PBEF transcript abundance was normalized.

NAD⁺ analysis was determined by HPLC. Cellular nucleotides were extracted using perchloric acid, neutralized with KOH, and stored at −80° C. (30). The deproteinized cell lysate residues were then analyzed by HPLC using a mobile phase of 10 mM KH₂PO₄, 0.12% di-n-butylamine, pH, 3.0. Sample was injected onto a Prodigy C8 column (150×3.2 mm, 5 μm) (Phenomenex, Torrance, Calif., USA) by a Hewlett Packard 1090 chromatograph. The column temperature and flow rate were maintained at 40° C. and 0.5 ml/min, respectively. The effluent was monitored at 260 nm by a Hewlett Packard 1050 UV-VIS detector and NAD⁺ retention time, determined from an NAD⁺ standard, was 10 minutes.

Histone Deacetylase (HDAC) Assays were employed. Histone H4 peptide (Upstate) was labeled with [³H]-acetyl Coenzyme A (ICN) using PCAF histone acetylase. Cell lysates were harvested using Passive Lysis Buffer (Promega) and 25 μg of protein was incubated at 37° C. for 6 hours with 50,000 cpm of [³H]-acetyl histone and 1 mM PMSF in HDAC Assay Buffer (Upstate). Released [³H]-acetate was extracted with ethyl acetate (31) and counted on a Beckman LS3801 scintillation counter.

Human-Mouse Chimeric Angiogenesis in vivo was measured. HITC6 SMCs in M199 with 10% FBS and fibroblast growth factor-2 (FGF-2) were mixed with an equal volume of growth factor-reduced matrigel (BD Discovery Labware) yielding final concentrations of 5×10⁵ cells/ml and 250 ng/ml FGF-2. The cell-matrigel suspension (500 μl) was subcutaneously injected into the abdomen of mice with severe combined immunodeficiency syndrome (SCID). After 8 days, implants were harvested, fixed for 8 h in Tris-buffered zinc (32), and paraffin-embedded tissues were sectioned at 5 μm. After deparaffinization, endogenous peroxidases were quenched with 0.3% H₂O₂, nonspecific binding was blocked with 5% goat serum and sections were immunostained for h-caldesmon, calponin (clone hCP, Sigma), CD31 (BD Pharmingen, Bedford, Mass.), and GFP (BD Clontech, Palo Alto, Calif.). To simultaneously visualize mouse endothelial cells and human SMCs, sections were incubated overnight at 4° C. with biotinylated rat anti-mouse CD31 and bound antibody reacted with ABC reagent (Vector labs, Inc., Burlingame, Calif.) and diaminobenzidine (DAB, Vector). Tissue was then incubated with rabbit anti-GFP (1:200, BD Clontech, Palo Alto, Calif.) and bound primary antibody detected with alkaline phosphatase-conjugated goat anti-rabbit secondary antibody and visualized with red alkaline phosphatase substrate (Vector). Single and double immunolabeled sections were counterstained with Harris' hematoxylin.

Microscopy and Image Analysis were used to measure cell robustness and function. Cell images were collected using a Zeiss Axiovert S100 microscope (Carl Zeiss Microimaging Inc.) equipped with Hoffman Modulation Contrast Plan objectives and TCT condenser (Modulation Optics Inc.), cooled QICAM 12-bit Mono Fast 1394 camera (QImaging Inc.) and Northern Eclipse image analysis software (Empix Imaging Inc.). Histology images were acquired with an Olympus BX50 microscope with a BX-FLA illuminator, UPlanF1 objective lenses (Olympus Optical Co. Ltd.), cooled Retiga EXi Mono Fast 1394 camera (QImaging Inc.) and Northern Eclipse image analysis software. Linear image processing was done using Photoshop CS (Adobe Systems Inc.).

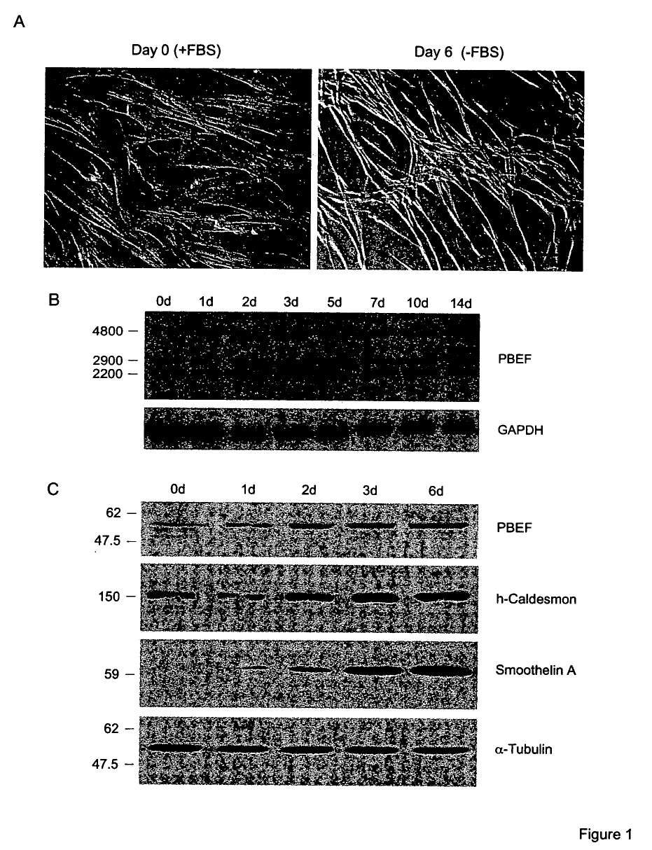

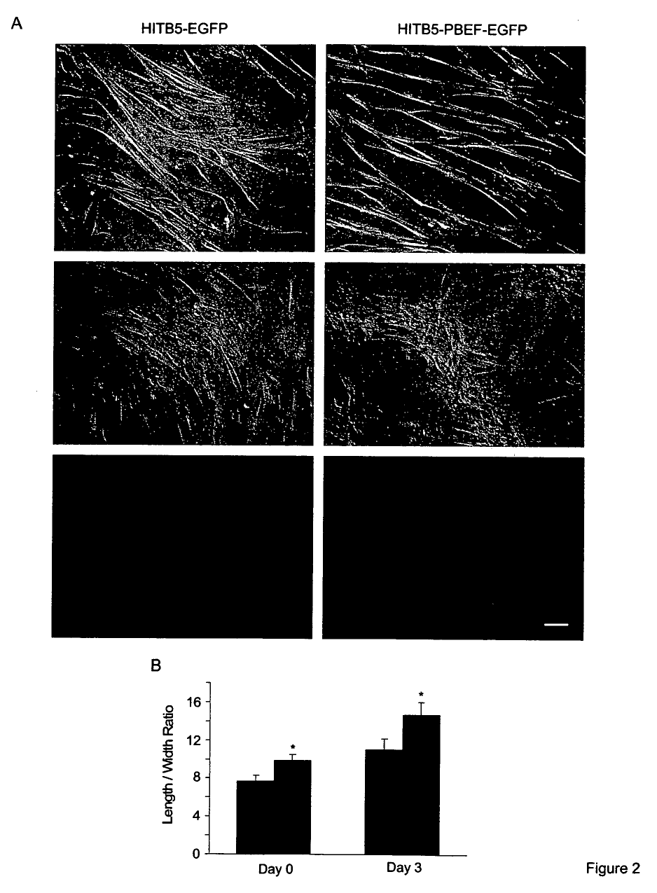

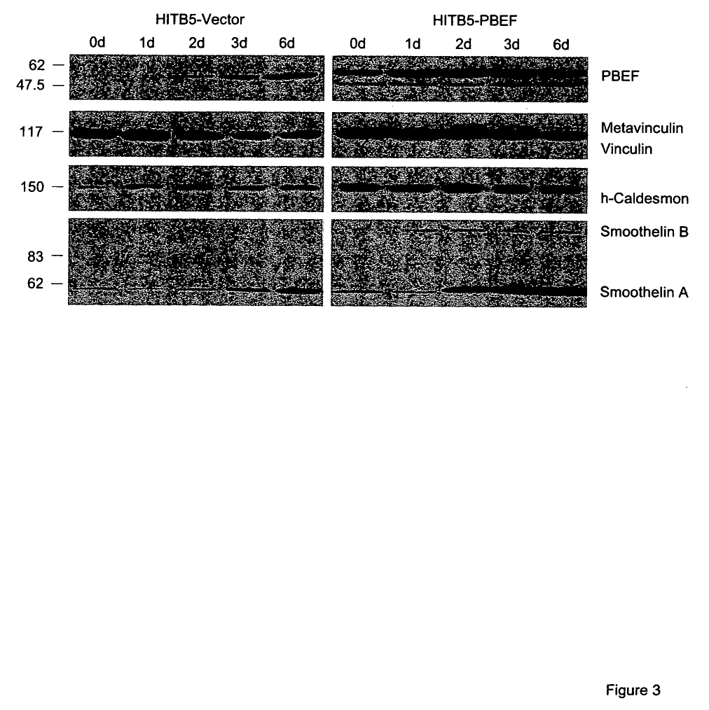

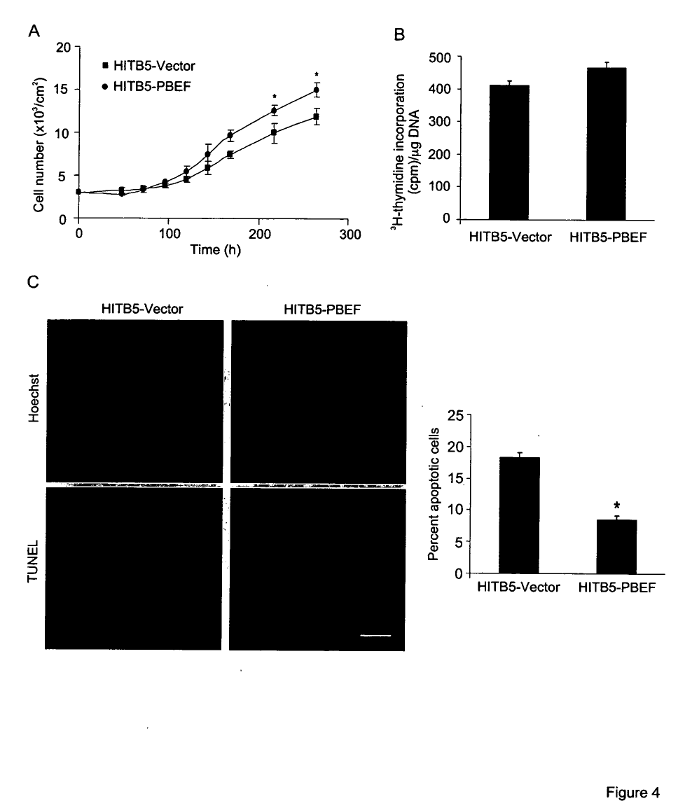

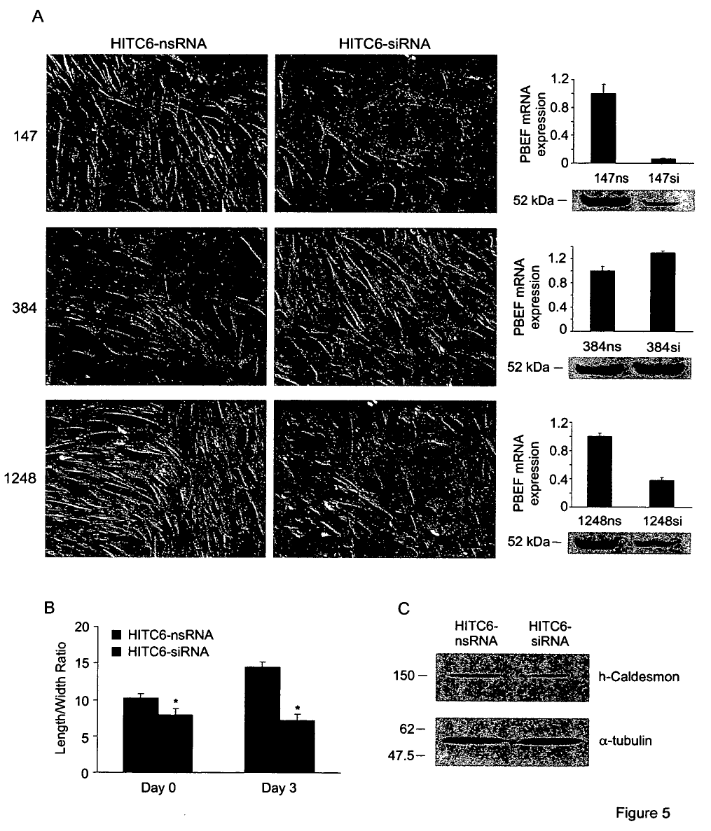

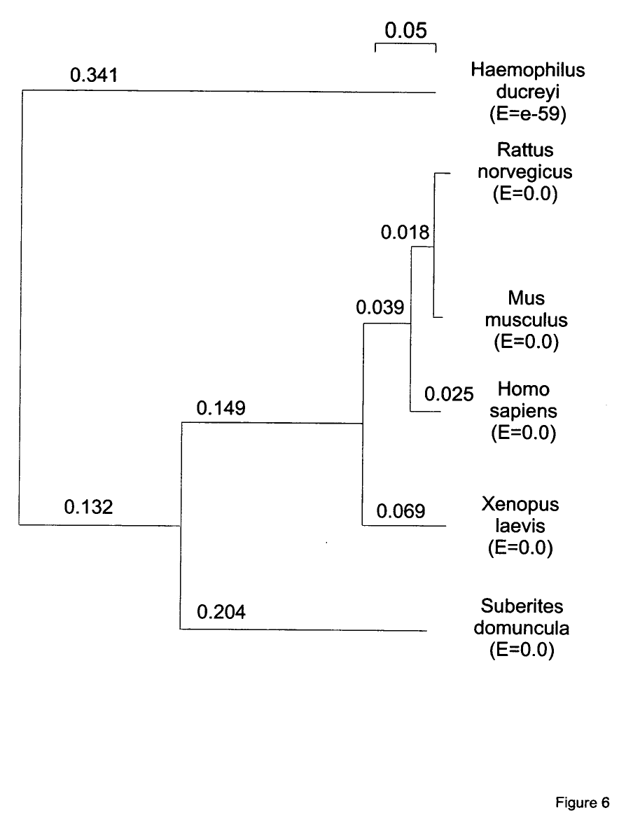

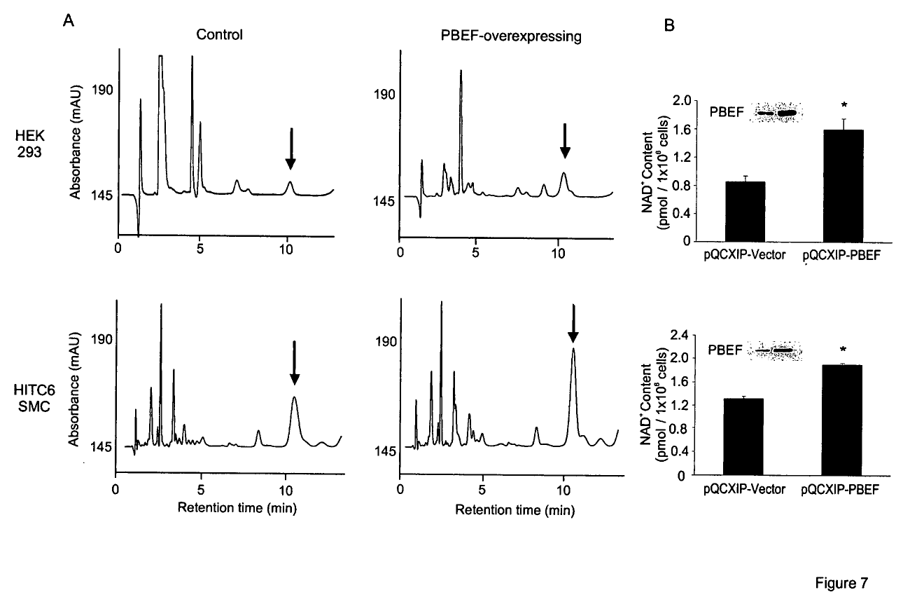

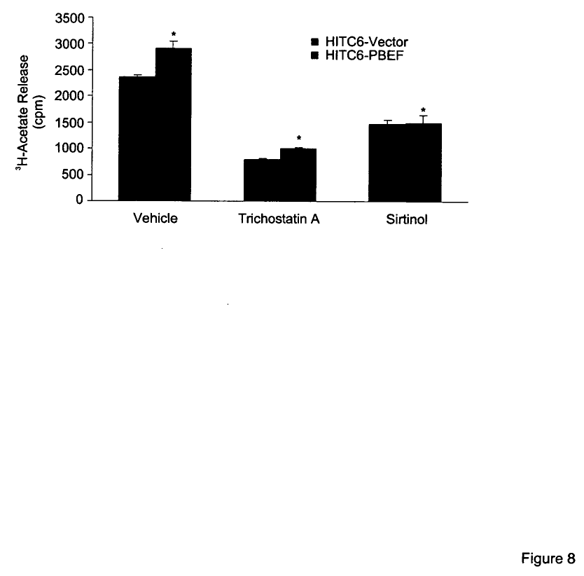

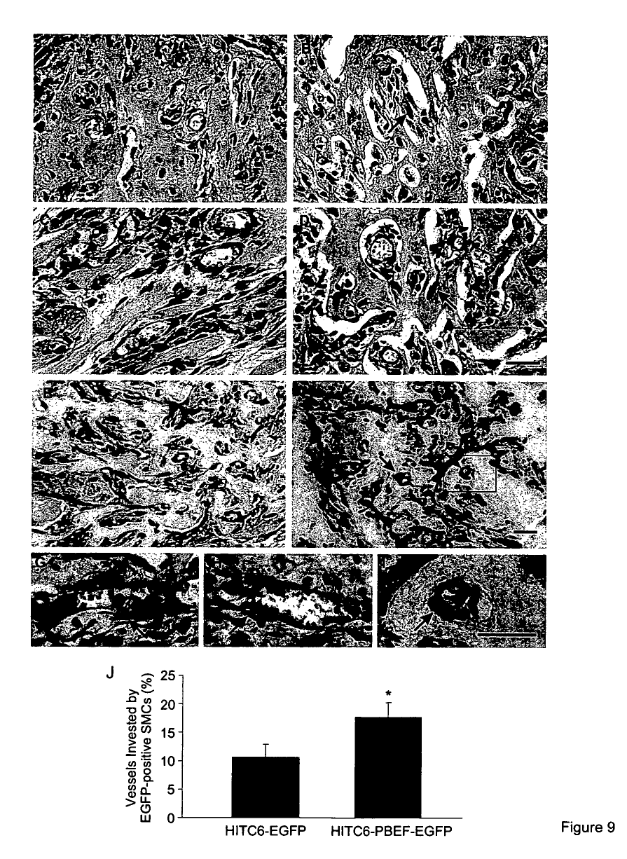

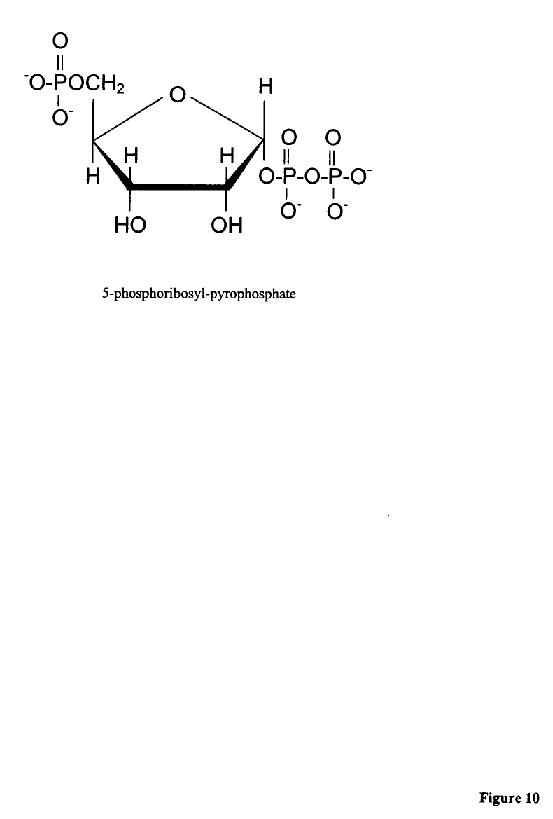

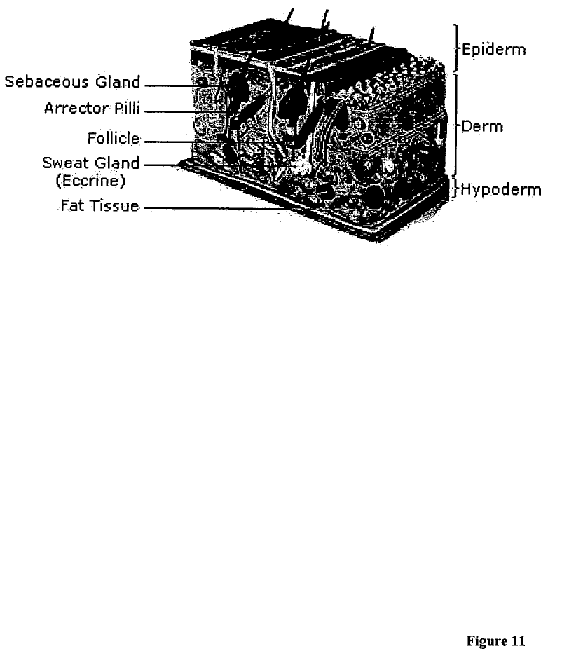

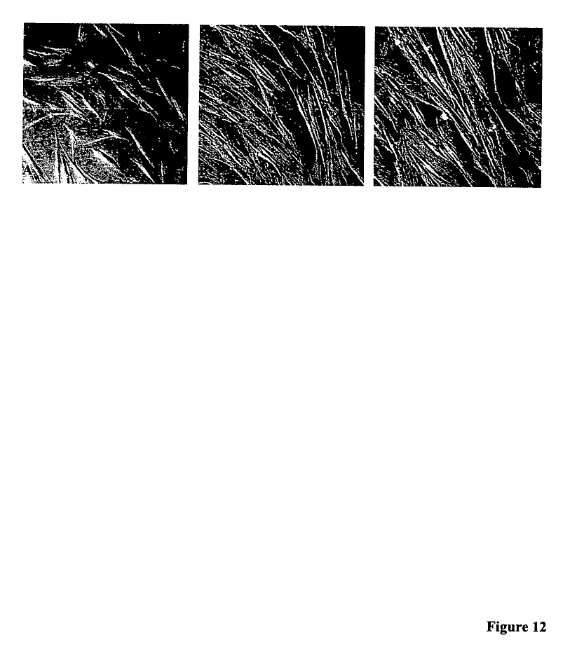

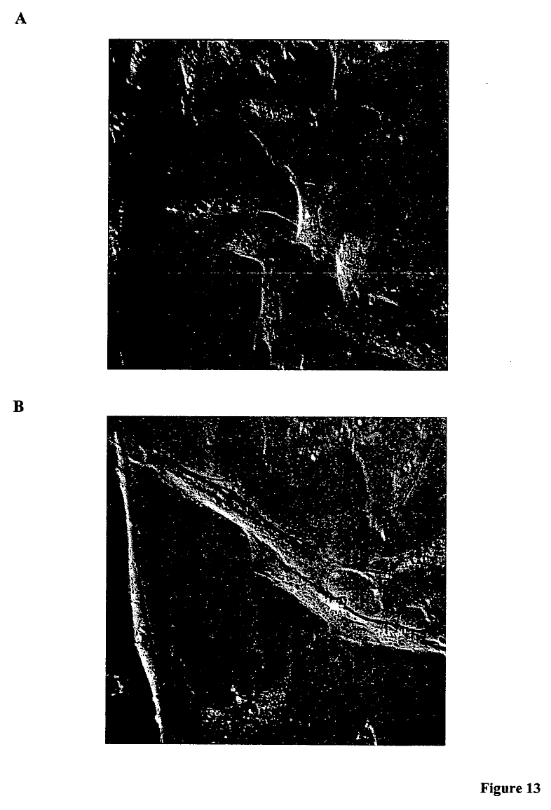

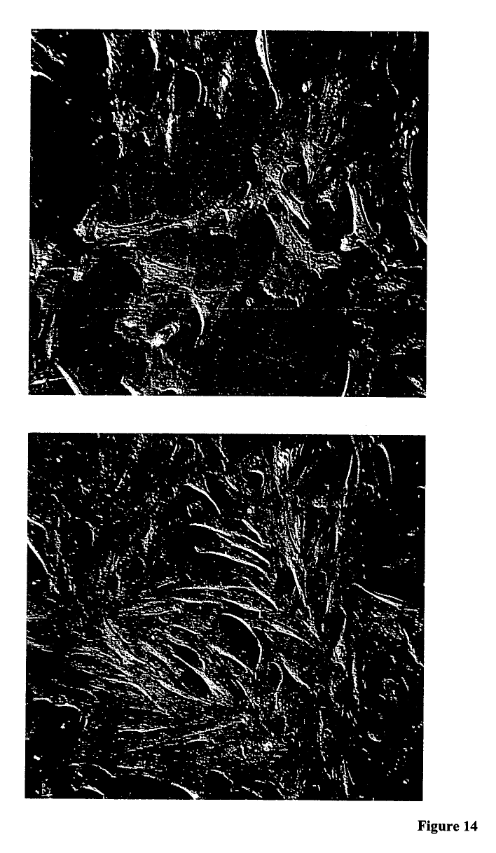

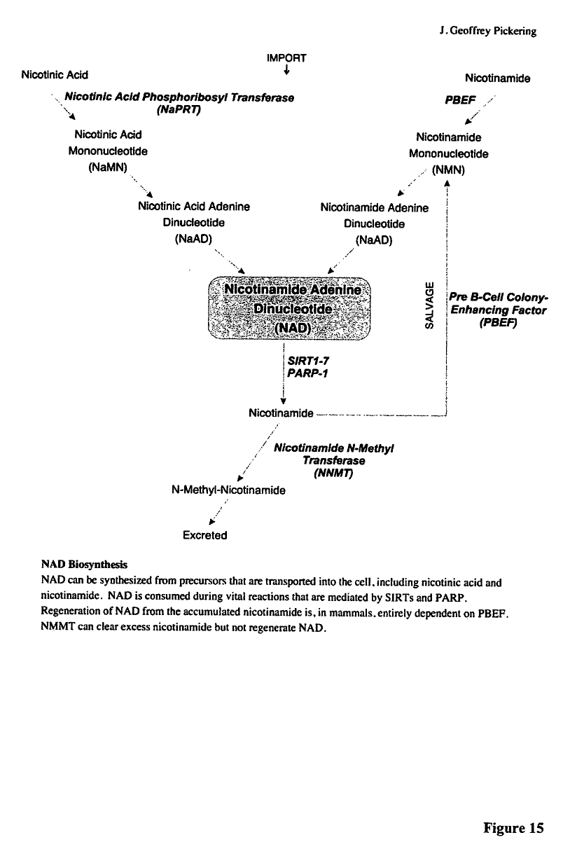

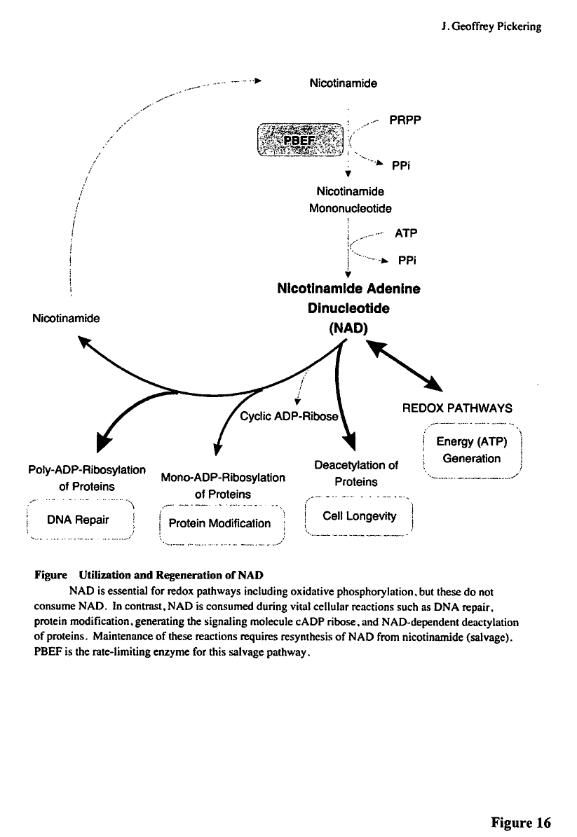

With respect to FIG. 1, we show that PBEF is upregulated during maturation of HITB5 and HITC6 human vascular SMC's. FIG. 1(A) shows Hoffman-modulated contrast images of HITB5 smooth muscle cells in M199 supplemented with 10% FBS (left images) and 6 days after culturing SMC's in serum-free M100 (right image);

FIG. 1(B) is a Northern Blot showing up-regulation of the 3 major transcripts of PBEF in HITB5 SMC's following withdrawal of serum from cultures. FIG. 1(C) is a Western blot of cell lysates harvested from HITC6 SMC's before and after withdrawal of serum from cultures. Expression of PBEF protein increases as does expression of the SMC differentiation markers, h-caldesmon and smoothelin A.

With respect to FIG. 2, we show that overexpression of PBEF in human SMC's induces cellular elongation and aggregation of cells into multilayered ridges. The images of FIG. 2 show the cellular elongation and aggregation of human Smooth Muscle Cells into multilayered ridges induced by the overexpression of PBEF. FIG. 2(A), shows Hoffman-modulated contrast images of sub-confluent (top panel) or post-confluent (middle panel) HITB5 Smooth Muscle Cells transduced with a retrovirus containing cDNA encoding EGFP alone (left image) or PBEF and EGFP from a bicistronic cassette (right image). The bottom panel of FIG. 2(A) depicts fluorescence images of the post-confluent SMC cultures, showing expression of the transgenes as indicated by EGFP fluorescence. Bar, 50 μm. FIG. 2(B) shows the quantification of length-width ratios of control and PBEF-overexpressing Smooth Muscle Cells cultured in the presence of serum and 3 days after serum withdrawal. The dimensions of 50 randomly selected SMC's were determined using Northern Eclipse software (*p<0.01 vs HITB5-EGFP SMC's).

With respect to FIG. 3, we show that overexpression of PBEF in human SMC's stimulates expression of SMC differentiation proteins. FIG. 3 shows Western blots revealing expression of SMC differentiation markers in HITB5 SMC's infected with cDNA encoding EGFP alone (left image), and cDNA encoding both PBEF and EGFP (right image). Transductants were selected with G418 and lysates harvested before and on the designated days after serum withdrawal. Blots for control and PBEF-overexpressing SMC's were probed with a given antibody and exposed simultaneously.

With respect to FIG. 4, we demonstrate that PBEF reduces SMC apoptosis. FIG. 4(A) shows cell accumulation over 11 days for control and PBEF-overexpressing HITB5 SMC's, cultured in M199 with 5% FBS. Cell numbers from quadruplicate wells were quantified using a hemacytometer and the result shown is representative of 2 separate experiments (*p<0.01). FIG. 4(B) shows Thymidine incorporation into control and PBEF-overexpressing HITB5 SMC's, assessed by incubating cells in log-phase growth with 10 μCi/mL [³H]thymidine for 12 hours. Thymidine incorporation is expressed relative to cellular DNA content, determined by fluorescence spectrometry of Hoechst 33258-stained lysates. FIG. 4(C) shows fluorescence images of control and PBEF-overexpressing SMC's stained with Hoechst 33258 to identify nuclei (top panel) and for apoptotic nuclei by incubating with d-UTP fluorescein (bottom panel). SMC's were plated on glass slides, cultured in M199 with 10% FBS, and fixed with 4% paraformaldehyde. *p<0.01 vs control HITB5 SMC's.

With respect to FIG. 5, we demonstrate that knock-down of PBEF expression reduces the viability of Smooth Muscle Cells, and prevents serum withdrawal-induced maturation. The images of FIG. 5 show the effect on SMC viability of the knockdown of PBEF expression and maturation induced by serum withdrawal. FIG. 5(A) shows Hoffman-modulated images of control HITC6 SMC's and HITC6-siRNA SMC's. Western blots showing PBEF protein expression for each cell line are shown. HITC6 SMC's were transduced with the pSIREN-RetroQ vector containing a non-silencing oligodeoxynucleotide (HITC6 nsRNA) or an oligodeoxynucleotide encoding a hairpin siRNA fragment (HITC6siRNA). Fragments were 19 nucleotides in length beginning at nucleotides 147, 384, and 1248 from the start of the coding sequence. Transductants were selected with puromycin. FIG. 5(B) shows the length-width ratios of 50 randomly selected cells expressing either nsRNA 1248 or siRNA 1248. B. Length-width ratios were determined for cells in M199 with 10% FBS and 3 days after serum withdrawal. *p<0.05 vs control SMC's expressing the non-silencing RNA. FIG. 5(C) is a Western blot showing reduced expression of hcaldesmon in HITC6-siRNA 1248 SMC's.

FIG. 6 shows that human PBEF is a phylogenetically conserved nicotinamide phosphoribosyltransferase. FIG. 6 depicts a phylogenetic tree showing a tight evolutionary relationship between bacterial nicotinamide phosphoribosyltransferases and eukaryotic PBEF, including human PBEF. Sequences bearing similarity to the full length H. sapiens PBEF protein were used to generate a phylogenetic tree which establishes a tight evolutionary relationship between bacterial nicotinamide phosphoribosyltransferases and eukaryotic PBEF, including human PBEF. A representative selection of organisms were chosen for illustration and expect (E) values for the designated sequence alignments are shown. The point accepted mutation matrix (PAM) distances between the leaves and nodes of the phylogenetic tree are also shown. Sequences depicted are: H. sapiens (g|1172027); M. musculus (g|113278525); R. norvegicus (gi|29293813); X. laevis (gi|28278775); S. domuncula (gi|6689202); H. ducreyi (gi|33152518).

The depictions of FIG. 7 show the effects of increasing PBEF levels on the levels of NAD⁺. FIG. 7(A) shows the HPLC analyses of deproteinized nucleotide extracts obtained from HEK293 cells transfected with pQCXIP or pQCXIP-PBEF, and HITC6 SMC's transduced with pQCXIP or pQCXIP-PBEF. NAD⁺, eluted from the column after approximately 10 minutes, is indicated by the arrow on the chromatograms. Quantitative data from 3 separate experiments for each cell type are shown in FIG. 7(B). Representative PBEF expression for control and PBEF-overexpressing cells is shown in the Western blot insets. The left lane of each blot depicts the control SMC's. *p<0.01 vs control cells.

FIG. 8 shows that increasing the levels of PBEF, increases NAD⁺-dependent histone deacetylase activity in human Smooth Muscle Cells. HITC6 SMC's were stably transduced with pQXCIP-PURO or pQXCIP-PBEF-PURO and HDAC activity determined in cell lysates (25 μg of protein), using [³H]histone H4 peptide as the substrate. The reactions were quenched by acid hydrolysis and catalytically released [³H]-acetate was extracted in ethyl acetate. Deacetylation reactions were performed in the presence of vehicle, 50 μM sirtinol, or 40 nM trichostatin A. *p<0.01 vs vector-infected SMC's under the same assay conditions.

With respect to FIG. 9, Smooth Muscle Cells overexpressing PBEF maintain a mature phenotype in vivo and promote vessel chimerism and SMC investment. FIGS. 9(A) through 9(D) show sections of matrigel implants loaded with human SMC's. FIGS. 9(E) through 9(I) show sections stained with h-caldesmon and h-calponin. FIG. 9(J) illustrates the quantification of the proportion of microvessels invested by at least one EGFP-positive SMC.HITC6 SMC's were stably transduced with pLNCX2-EGFP or pLNCX2-PBEF-EGFP, mixed with matrigel and 250 ng/ml FGF-2 and transplanted beneath the skin of SCID mice. The matrigel implants were harvested 8 days later and paraffin-embedded sections studied by immunohistochemistry. FIGS. 9(A-D), show sections of matrigel implants that had been loaded with either control or PBEF-overexpressing human SMC's, immunostained for human hcaldesmon (A, B) or calponin (C, D). Implants are populated by newly formed microvessels and xenotransplanted human SMC's. Staining of h-caldesmon and hcalponin is more prominent in implants containing PBEF-overexpressing SMC's (arrows). In FIG. 9(E-I), sections of zinc-fixed matrigel implants double-immunolabeled for endothelial cells (anti-mouse CD31) and human SMC's (anti-GFP). Bound anti-CD31 antibody was identified using DAB chromogen (brown color) and bound anti-EGFP antibody was visualized using red alkaline phosphatase substrate (red color). A proportion of newly formed blood vessels are invested by exogenously added human SMC's and this is especially prominent for implants containing PBEF-overexpressing SMC's (arrows). FIGS. 9(G-I) show high-magnification images showing intimate apposition of EGFP-positive, PBEFoverexpressing SMC's with mouse endothelial cells. In FIG. 9(G), a human SMC is shown aligned parallel to an endothelial cell-lined vessel containing red blood cells and leukocytes. FIG. 9(H) shows the partial contact of an elongated human SMC with an endothelial cell, possibly reflecting active investment of the microvessel by the transplanted SMC. In FIG. 9(I), which corresponds to the box in F, a human SMC is circumferentially wrapped around the mouse microvessel. All sections were counterstained with Harris' hematoxylin. Bar, 50 μm. J. Quantification of the proportion of microvessels invested by at least one EGFP-positive SMC. (*p<0.05 vs HITC6-EGFP-loaded gels).

FIGS. 12 A-C are phase contrast photomicrographs, at 20× magnification, of vector-transduced or PBEF-overexpressing HITC6 SMC's in response to treatment with 5-phosphoribosyl-pyrophosphate (PRPP), demonstrating that the PRPP increases the health of smooth muscle cells compared to control cells. FIGS. 12(A-C) show cells infected with vector HITC6 with vehicle that have been cultured in M199 containing 1% FBS for 24 hours prior to addition of 250 uM PRPP. In FIG. 12(A), HITC6-infected SMC's with vehicle have been cultured in 1% FBS for 72 hours. In FIG. 12(B), the SMC's have been cultured with PBEF plus PRPP. In FIG. 12(C), the SMC's have been cultured with PBEF plus PRPP plus 1 mM Nam.

FIGS. 13 A-B are phase contrast photomicrographs at 20× magnification of vector-transduced HITC6 SMC's in response to treatment with 5-phosphoribosyl-pyrophosphate (PRPP), demonstrating that the PRPP increases the health of smooth muscle cells compared to control cells. The cells were cultured were cultured in M199 containing 1% FBS for 24 hours prior to addition of 500 uM PRPP. FIG. 13(A) shows the results of HITC6-Vector with vehicle at 24 hours. FIG. 13(B) shows the results of HITC6-Vector plus PRPP at 24 hours.

FIGS. 14 A-B show the protective effects of providing Smooth Muscle Cells with PRPP. After 24 hours in a stressing low serum environment, cells given PRPP alone are protected from cell death and are generally more robust. FIGS. 14 A-B are phase contrast photomicrographs at 10× magnification of vector-transduced HITC6 SMC's in response to treatment with 5-phosphoribosyl-pyrophosphate (PRPP). SMC's were cultured in M199 1% FBS for 24 hours prior to addition of 500 uM PRPP. FIG. 14(A) shows the results of HITC6-Vector with vehicle at 24 hours. FIG. 14(B) shows the results of HITC6-Vector plus PRPP at 24 hours.

Pre-B Cell Colony Enhancing Factor is Upregulated During SMC Maturation. SMC maturation entails the final stages of the SMC developmental program and confers cells with the capacity to contract. The generation of clonal populations of SMC's from the human internal thoracic artery, designated HITB5 and HITC6, which can convert from a proliferative state to a contractile SMC (5, 6), enabled us to screen for endogenous factor involved in this phenotype conversion.

The Smooth Muscle Cell lines designated HITB5 and HITC6, in contrast to other vascular preparations, are capable of reversibly converting their phenotype between a spread, noncontractile state in the presence of serum and an elongated, contractile state after serum withdrawal (Li, Circ. Res, 1999). The withdrawal of serum induces in these cells a stress response, one that requires the cells to adapt to ensure survival. Primary cultures of SMC's cultured from explants are generally incapable of acquiring a truly, differentiated state in such stress conditions, and display an increased apoptotic rate. One of the hallmarks of HITB5 and HITC6 SMC's is their ability to adapt to media that is serum-free, suggesting that they possess the capability to activate a stress response that is sufficient to deal with the decreased abundance of nutrients and mitogens. Li, S., Sims, S., Jiao, Y., Chow, L. H., and Pickering, J. G. Evidence From a Novel Human Cell Clone That Adult Vascular Smooth Muscle Cells Can Convert Reversibly Between Noncontractile and Contractile Phenotypes. Circulation Research. 85(4): 338-348, 1999.

These screens suggested that PBEF was substantially upregulated as SMC's adopted a mature, contractile state. To verify this finding, HITB5 SMC's were analyzed for PBEF mRNA and protein expression by Northern and Western blot analysis. As shown in FIG. 1, six days after serum withdrawal, HITB5 SMC's converted from spread cells variably oriented on the dish to highly elongated cells that had crawled in a directed fashion into multilayered cell aggregates. Concurrently, the three major transcripts of PBEF (4.8, 2.9, and 2.2 kb) were substantially upregulated (FIG. 1b). Intracellular PBEF protein abundance also increased in maturing HITB5 and HITC6 SMC's, as did the expression of the contractile apparatus proteins, h-caldesmon and smoothelin A (FIG. 1c), confirming the relationship between PBEF expression and SMC maturation. PBEF was not detected in concentrated culture media at any stage of the maturation program (data not shown).

Overexpression of PBEF stimulates the maturation of smooth muscle cells. To determine if PBEF was functionally linked to SMC phenotype, we overexpressed PBEF in immature human SMC's. HITB5 SMC's were infected with retrovirus containing either pLNCX2-IRES-EGFP (HITB5-EGFP) or pLNCX2-PBEF-IRES-EGFP (HITB5-PBEF) and stable transductants were selected with neomycin. Under baseline, serum-supplemented conditions, SMC's overexpressing PBEF were longer and thinner than vector-infected SMC's. Elongation in response to PBEF was also observed with HITC6 SMC's and primary SMC's (data not shown). Furthermore, elongation of PBEF-overexpressing SMC's relative to control SMC's persisted as the cells further elongated in response to serum withdrawal (FIG. 2). To determine if the spatial organization of maturing SMC's was impacted by PBEF, cells were plated at higher densities (12,000 cells/cm²) and subjected to serum withdrawal. After 3 days, control SMC's had begun to aggregate although the extent of patterning was modest. In contrast, HITB5-PBEF-EGFP SMC's rapidly aggregated and by 3 days had already assembled into discrete, multi-layered ridges and nodules (FIG. 2).

To assess the effect of PBEF on SMC contractile protein expression, cells were studied by Western blot analysis. As shown in FIG. 3, HITB5-EGFP SMC's displayed the characteristic upregulation of PBEF following serum withdrawal, together with increased expression of h-caldesmon and smoothelin A. Interestingly, PBEF-overexpressing HITB5 SMC's displayed increased levels of h-caldesmon and smoothelin A under baseline, serum-supplemented conditions, assessed from cell lysates probed and exposed simultaneously with that of vector-infected SMC's. As well, smoothelin B, which was not detected in control SMC's, was expressed in PBEF-overexpressing SMC's. Withdrawal of serum from cultures of HITB5-PBEF SMC's lead to further upregulation of smoothelin A. Moreover, metavinculin expression was induced following serum withdrawal from PBEF-overexpressing SMC's but remained undetected in HITB5-EGFP SMC's. Thus, augmented expression of PBEF shifts the morphological and biochemical phenotype of SMC's closer to that of mature SMC's in the adult vessel wall. This similarity to contractile SMC's in vivo was especially strong when serum was removed from the culture environment.

Overexpression of PBEF reduces the degree and extent of SMC apoptosis. To determine whether SMC growth was impacted by PBEF expression, SMC's in 5% serum were tracked over an 1′-day period. As shown in FIG. 4(A), HITB5-PBEF SMC's accumulated faster than HITB5-EGFP SMC's, with a doubling time of 5.4 days versus

7.3 days, respectively (p<0.01). Similar results were seen with primary cultures of SMC's overexpressing PBEF (data not shown). To determine if this increase in cell accumulation was due to increased DNA synthesis, SMC's were incubated with ³H]thymidine and thymidine incorporation, relative to total DNA content, was assessed.

As shown in FIG. 4b, there was no detectable difference in thymidine incorporation between control and PBEF-overexpressing cells. To assess if SMC survival was affected by PBEF, apoptosis was assessed using TUNEL. This revealed that the proportion of apoptotic HITB5-PBEF SMC's was approximately half that of HITB5-EGFP SMC's (p<0.01, FIG. 4c). This improved survival was consistent with the appearance of PBEFoverexpressing SMC's, including their smoothly contoured cell surface and the paucity of culture debris.

siRNA-mediated PBEF knockdown impairs the survival and maturation of SMC's. We next determined if endogenous PBEF was required for SMC maturation. For this, HITC6 SMC's were infected with retrovirus containing cDNA encoding a hairpin-forming siRNA fragment. To ensure the siRNA responses reflected PBEF knockdown, 3 different targeting fragments were studied and both PBEF mRNA and protein were quantified. Two of the 3 siRNA constructs (siRNA147, siRNA1248) yielded a significant decrease in PBEF mRNA, quantified by real-time RT-PCR, compared to control SMC's infected with retrovirus containing cDNA encoding the corresponding non-silencing, RNA fragment (nsRNA147, nsRNA1248). SMC's expressing siRNA147 or siRNA1248 also showed significant suppression of PBEF protein. These SMC's had a short, truncated morphology (FIG. 5a) and they survived poorly, precluding serial passages. As well, the small fraction of PBEF-knockdown SMC's that remained adhered to the culture dish did not elongate following serum withdrawal (FIG. 5b). Heavycaldesmon expression was also significantly lower in PBEF-knockdown SMC's than control SMC's (FIG. 5c). The limited cell viability precluded assessing h-caldesmon expression in response to serum withdrawal. Overall however, the poor cell survival, the perturbed morphology and inability to elongate, and the low expression of h-caldesmon in surviving SMC's indicate an inability of PBEF-knockdown SMC's to mature in culture. In contrast, SMC's expressing the siRNA construct (siRNA384) that did not manifest a reduction in PBEF mRNA or protein maintained an elongated morphology (FIG. 5a) and responded to serum withdrawal normally.

PBEF Increases Intracellular NAD⁺. In view of the controversy over human PBEF function and whether it acts intracellular or extracellularly, we generated a phylogenetic tree using protein sequences similar to human PBEF, derived from a Blast search of the NCBI sequence database. Multiple sequence alignment generated using ClustalW revealed that sequences from diverse species, bacterial and eukaryotic, were similar in length, contained strongly conserved regions with respect to helix propensity and hydrophobicity, and a conserved phosphoribosyltransferase domain. As shown in FIG. 6, the phylogenetic tree using selected sequences revealed very low point-assisted mutation scores, reflecting the short distances amongst all nodes and leaves of the tree. Thus, the PBEF protein has been well conserved throughout evolution which suggests a fundamental and invariant role. As this role has been shown in bacteria and rodent cells to involve NAD⁺ biosynthesis (10, 12), we next determined if the level of NAD⁺ in human cells was affected by PBEF. Analysis of cellular nucleotides by HPLC revealed that NAD⁺ content in HEK293 cells stably expressing the PBEF transgene was significantly higher than control cells (1.60±0.15 vs 0.85±0.08 pmol/10⁶ cells, p<0.01). Likewise, NAD⁺ content in HITC6-PBEF SMC's was greater than that in HITC6-Vector SMC's (1.90±0.02 vs 1.31±0.04 pmol/10⁶ cells, p<0.01) (FIG. 7).

NAD⁺-Dependent HDAC Activity is Increased in SMC's Overexpressing PBEF. Having established that PBEF increases intracellular NAD⁺ content in SMC's, we considered how this might impact SMC performance. NAD⁺-consuming reactions that depend on NAD⁺ regeneration include the deacetylation of certain histones and other proteins, postranslational modifications critical to gene silencing and cell survival (16, 17). To determine if PBEF influenced histone-deacetylase (HDAC) activity in SMC's, lysates from control and PBEF-overexpressing SMC's were incubated with [³H]-acetylated histone H4 peptide and HDAC activity quantified. As shown in FIG. 8, total HDAC activity was significantly greater in HITC6-PBEF SMC's than HITC6-Vector SMC's. To determine the relative amount of NAD⁺-dependent HDAC activity, deacetylase reactions were performed in the presence of 50 μM sirtinol, a noncompetitive inhibitor of NAD⁺-dependent (Class III) HDACs. Sirtinol significantly inhibited HDAC activity in PBEF-overexpressing SMC's, with a more modest inhibition of HDAC activity in control SMC's such that there was no longer a difference in HDAC activity between control and PBEF-overexpressing cells. We also examined the effect of 40 nM trichostatin A (TSA), an inhibitor of Class I and II HDACs, but not the NAD⁺-dependent HDACs. This substantially inhibited HDAC activity in both control and PBEF-overexpressing SMC's. However, the residual TSA-independent HDAC activity remained significantly greater in PBEF-overexpressing SMC's than in control SMC's. Taken together, the findings indicate that PBEF increases HDAC activity in human vascular SMC's and most, if not all, of this increase can be attributed to NAD⁺-dependent HDAC activity.

SMC's overexpressing PBEF Invest Newly Formed Blood Vessels In Vivo. In order to determine if the survival and maturation profile observed in PBEF-overexpressing SMC's in vitro could translate into enhanced SMC performance in vivo, we studied SMC-based remodeling of newly formed blood vessels. During angiogenesis, SMC's wrap around the nascent vessel and assume the specialized phenotype. This investment process both stabilizes the microvessel and provides the machinery for vasomotor control (3, 18). To assess the integration of SMC's into the nascent vasculature, we developed a human-mouse chimeric model of angiogenesis. Growth factor-reduced matrigel mixed with FGF-2 and either PBEF-overexpressing (HITC6-PBEF-EGFP) or vector-transduced HITC6 SMC's (HITC6-EGFP) was injected subcutaneously into the abdominal regions of SCID mice. After eight days, mice were sacrificed and zinc-fixed, paraffin-embedded sections were studied histologically. By design, most of the interstitial cells in the implants were xenotransplanted human SMC's. Compared to control human SMC's, PBEF-overexpressing SMC's displayed greater immunoreactivity for the SMC maturation markers, h-caldesmon and calponin (FIG. 9(A)-(D)). Vascular integration of transplanted SMC's was determined by doubleimmunolabeling for mouse endothelial cells (anti-CD31) and human SMC's (anti-EGFP). Immunostaining for EGFP, as opposed to assessing EGFP fluorescence, proved to be a more sensitive detection system for SMC's in paraffin-embedded tissue. Paraffin processing, in turn, was critical to maintaining tissue architecture so that investment of endothelial-lined channels by SMC's could be unequivocally determined. As illustrated in FIG. 9e-i, a small proportion (approximately 2-5%) of human SMC's associated with endothelial cells. SMC's that invested microvessels assumed an elongated morphology with a more compact and elongated nucleus compared to SMC's in the interstitium. The proportion of microvessels that were invested by one or more human SMC's was significantly higher in matrigel implants containing PBEF-overexpressing SMC's (17.8±2.5%) than in implants loaded with vector-infected SMC's (10.7±2.2%, p<0.05) (FIG. 9j). PBEF-overexpressing SMC's could be found aligned with the long axis of microvessels, partially apposed to the endothelial cell as if actively extending to form the wall, or wrapped circumferentially around the microvessel (FIG. 9). Thus, SMC's with augmented capacity for NAD⁺ biosynthesis responded to the angiogenic environment in a specialized manner and integrated into the vasculature more efficiently than control SMC's.

SMC's in developing arteries, and a proportion of SMC's in diseased adult arteries, exist in an immature, non-contractile state. To mature into a phenotype found in the normal adult artery wall, these cells must exit the cell cycle, elongate, and acquire the capacity to contract in response to vasoactive stimuli. We have shown that this switch in SMC phenotype depends on the actions of PBEF. PBEF was substantially upregulated as human SMC's converted to the contractile state and was essential for the survival of SMC's in an environment that no longer supported SMC proliferation. Furthermore, increased expression of PBEF stimulated cellular elongation, increased the expression of multiple SMC marker genes, and promoted vascular maturation in vivo. This augmentation in SMC maturation by PBEF was associated with increased intracellular levels of intracellular NAD⁺ and increased NAD⁺-dependent histone deacetylase activity. Collectively, these findings implicate PBEF-mediated NAD⁺ flux as a regulatory system for SMC phenotype.

The SMC differentiation program is characterized by the ordered expression of genes that encode proteins of the SMC contractile apparatus (2). The HITB5 and HITC6 SMC's used in this study express a number of these proteins, even in the proliferative, noncontractile phenotype, including smooth muscle α-actin, h-caldesmon, and smoothelin A (5). Thus, the shift to a contractile state for these cells, induced by serum withdrawal, represents a late phase of the SMC developmental program. Primary human SMC cultures typically are not capable of this shift and also do not survive in the absence of serum or exogenous survival factors. In addition, expression of PBEF is lower in primary human SMC cultures than in the maturation-competent clones used in this study (data not shown). In this context, our finding that SMC viability was compromised by PBEF knockdown implies that PBEF is, as a minimum, permissive for SMC maturation. That is, PBEF may ensure the cell's survival in an environment that does not support cell growth. In addition, overexpression of PBEF yielded elongated SMC's with elevated levels of h-caldesmon, smoothelin A, and smoothelin B, even in the presence of serum, suggesting that PBEF also participates more directly in the maturation program. Following serum withdrawal, SMC maturation was even more robust, evidenced by expression of metavinculin and the striking multicellular patterning. Thus, PBEF may influence SMC differentiation by both facilitative and stimulatory mechanisms. These actions may also be relevant to other cell types because upregulation of PBEF expression has been observed during maturation of dendritic cells and B-lymphocytes (7, 19, 20).

The molecular pathways by which PBEF acts have been controversial. PBEF was initially identified during a screen for cytokines and proposed to be a cytokine based on sequences in the 3′ untranslated region and on effects of recombinant PBEF on the formation of pre-B-lymphocyte colonies. In these studies, recombinant PBEF by itself did not influence colony formation and the effect was observed only after addition of stem cell factor and IL-7 (7). Support for the notion of PBEF as a cytokine has come from studies wherein recombinant PBEF, generated by bacteria, stimulated the expression of inflammatory genes (8, 21) and inhibited neutrophil apoptosis (9). Whether these effects were mediated by a cytokine receptor is unclear. Moreover, the assignment of PBEF as a cytokine has been challenged because PBEF has no coding sequence homology to cytokines, no signal sequence for secretion, and does not appear to be secreted from cells by either a classical or alternative pathway (10, 11). We were unable to detect PBEF protein in the concentrated conditioned media from PBEF-overexpressing cell lines despite substantial upregulation of intracellular protein.

In the alternative, the inventors theorize that PBEF is a nicotinamide phosphoribosyltransferase, the rate-limiting, intracellular enzyme for generating NAD⁺ from nicotinamide (10). The sequence analyses and phylogenetic tree described herein are entirely consistent with the identity of human PBEF as a nicotinamide phosphoribosyltransferase. This assignment of PBEF is further strengthened by our finding that expression of PBEF increases the intracellular level of NAD⁺ in both HEK293 cells and human vascular SMC's. We propose that the sequence data, the close evolutionary relationship with prokaryotic orthologs, the fact that PBEF upregulates NAD⁺, and previous complementation and enzyme analyses of mouse PBEF (10) bring the weight of evidence to the conclusion that human PBEF is a nicotinamide phosphoribosyltransferase and not a cytokine.

A number of vital enzymatic reactions, including deacetylation of histones, utilize and consume NAD⁺ (22). Degradation of NAD⁺ in these non-redox reactions liberates nicotinamide, from which NAD⁺ can be regenerated via a two-step salvage pathway. Nicotinamide phosphoribosyltransferase/PBEF catalyzes the conversion of nicotinamide to nicotinamide mononucleotide (NMN). NMN is then converted to NAD⁺ by nicotinamide mononucleotide adenylyl-transferase-1 (Nmnat1) (14, 15). Interestingly, Nmnat1 has recently been found to increase NAD⁺ synthesis and prevent axonal degeneration in explanted mouse neurons (23). Furthermore, NAD salvage pathway genes in yeast and mammals have been found to activate the NAD⁺-dependent HDAC, Sir2 (or the mammalian ortholog, SIRT1). Sir2 is a longevity enzyme that deacetylates H3 and H4 histones, certain transcription factors, and p53 (22, 24-26). The current findings with PBEF thus strengthen an emerging paradigm that NAD⁺ salvage impacts mammalian cell survival and they specifically link PBEF with NAD⁺-dependent protein deacetylation. Moreover, the data establish that the functional consequences of NAD⁺ salvage are not limited to cell survival but also strongly impact the SMC maturation program.

In order for newly formed vascular networks to survive, they must be ensheathed by SMC's for support and stabilization (3, 27). Investment by SMC's also enables the vessels to respond to vasoactive stimuli and thereby appropriately distribute blood. We capitalized on this in vivo, integrative response as a functional readout for SMC maturation. Human SMC's overexpressing PBEF retained their biochemical attributes of maturity when transplanted into mice and they productively responded to the angiogenic environment by integrating into the microvessel wall. This physiologic response not only confirms the positive impact of PBEF on SMC specialization but opens new possibilities for improving angiogenesis and arteriogenesis. SMC investment is very important for neovessels in ischemic tissues which are prone to regression. It has also been established that tumor vasculature is inadequately invested by SMC's, which can result in leaky vessels that are predisposed to tumor shedding (28). The current findings suggest that NAD⁺ biosynthesis pathways may be relevant to these clinical problems.

In summary, PBEF is not a cytokine but an intracellular protein that regulates NAD biosynthesis, NAD-dependent histone deacetylation, and vascular SMC maturation. Augmentation of NAD⁺ flux, via PBEF, could have therapeutic potential for vascular disease, angiogenesis, and tumor metastases.

Similar conclusions can be reached with respect to PRPP, and with respect to the effect of optimizing the concentration of intracellular NAD on protecting and rejuvenating skin.

The Skin, the body's largest organ, is a multi purpose organ that plays many important roles. Surprisingly, until the last 10 years, little has been known about the way the skin functions and its components. The surface of the skin is made of a conglomeration of dead cells. Underneath the surface, there are very thin and distinct layers, which are called the epidermis, the dermis and the hypodermis.

The Epidermis, which thickness varies from 0-04 mm to 1.6 mm, is an important layer. The Langerhans cells, responsible for the immunology of the skin, the melanocytes and tyrosinase enzyme, responsible for the production of melanin and pigment, are located in the epidermis. Thus, the epidermis is the skin layer that is responsible for both the look and the health of the skin. It protects the skin from moisture loss and the penetration of chemical products and bacteria and acts as the initial barrier to oxidant assault. Since it houses essential free radical scavengers such as vitamins E and C and super oxide dismutase many treatments for skin disorders include anti-oxidants to protect the skin cells from damage. We are disclosing the first treatment that enhances the cells metabolic fitness and increases cell longevity by allowing the cells to perform in an optimal manner.

This method of using PRPP alone or in combination with nicotinamide also serves as a cyto-protective purpose by reducing the amount of damage that a cell undergoes in times of stress. Maintaining a the health of the epidermal layer is vastly important, especially since they house Langerhans cells which are ultraviolet radiation (UVR) sensitive and even minor UVR exposure will damage the langerhans cells enough to reduce the skin's immune capacities. With age, these cells also decrease in number, one reason why the elderly have higher potential rates of skin disease. By increasing the longevity of cells by application of PPRP we can reduce the rates of skin disease by maintaining healthy cells.

The Dermis is the second layer of the skin and is 5 to 7 times thicker than the epidermis, it lies below the epidermis and is connected to it by the basement membrane.

The Dermis consists of a thick connective membrane criss-crossed by blood vessels, lymphatic vessels, nerve fibers and many sensory nerve endings. The Dermis provides nutrients to the epidermis though its vast network of capillaries and blood vessels and forms a supporting framework, composed of collagen and elastin protein fibers. It is primarily responsible for the skin's elasticity and acts as a water storage site. The dermis protects the body from mechanical injury and plays an important role in sensory perception and as an internal regulator and would benefit from the therapeutic effects of PRPP applications.

The inventors have also discovered that PRPP can be used to treat such diseases and conditions, including cancer, with low toxicity for normal healthy cells and can also be used to treat degeneration and inflammation.

In one embodiment, the disease state is cancer. Types of cancer that may be treated according to the present invention include, but are not limited to, hematopoietic cell cancers including leukemias and lymphomas, colon cancer, lung cancer, kidney cancer, pancreas cancer, endometrial cancer, thyroid cancer, oral cancer, ovarian cancer, laryngeal cancer, hepatocellular cancer, bile duct cancer, squamous cell carcinoma, prostate cancer, breast cancer, cervical cancer, colorectal cancer, melanomas and any other tumours. Solid tumours such as sarcomas and carcinomas include but are not limited to fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (such as a glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma).

The PPRP and or the nicotinamide molecules may be administered to the patient using standard methods of administration. In one embodiment, the molecule is administered systemically. In another embodiment, the molecule is administered by injection at the disease site. In a particular embodiment, the disease state is a solid tumour and the molecule is administered by injection at the tumour site. In various embodiments, the molecule may be administered orally or parenterally, or by any standard method known in the art.

When administered to a patient, an effective amount of PRPP is the amount required, at the dosages and for sufficient time period, to alleviate, improve, mitigate, ameliorate, stabilize, prevent the spread of, slow or delay the progression of or cure the disease.

Effective amounts of the molecule can be given repeatedly, depending upon the effect of the initial treatment regimen. Administrations are typically given periodically, while monitoring any response. It will be recognized by a skilled person that lower or higher dosages than those indicated above may be given, according to the administration schedules and routes selected.

The molecule may be administered alone or in combination with other therapies, including nicotinamide, PBEF, anti-oxidants, herbal and non-medicinal oils and substances.

To aid in administration, the molecule(s) may be formulated as an ingredient in a pharmaceutical composition. Therefore, in a further embodiment, there is provided a pharmaceutical composition comprising the PRPP and a pharmaceutically acceptable diluent. The compositions may routinely contain pharmaceutically acceptable concentrations of salt, buffering agents, preservatives and various compatible carriers. Generally, the pharmaceutical composition will be formulated with components that will not significantly impair the biological properties of the PRPP molecule.

The pharmaceutical composition can be prepared by known methods for the preparation of pharmaceutically acceptable compositions suitable for administration to patients, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985).

The pharmaceutical composition may be administered to a patient in a variety of forms depending on the selected route of administration, as will be understood by those skilled in the art. The composition of the invention may be administered topically, orally or parenterally. Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time.

The pharmaceutical composition may be administered orally, for example, with an inert diluent or with an assimilable carrier, or it may be enclosed in hard or soft shell gelatin capsules, or it may be compressed into tablets. For oral therapeutic administration, the PRPP molecule(s) may be incorporated with an excipient and be used in the form of ingestible tablets, buccal tablets, troches, capsules, elixirs, suspensions, syrups, wafers and the like.

In different embodiments, the composition is administered topically, by injection (subcutaneously, intravenously, intramuscularly, etc.) directly at the disease site, such as a tumour site, or by oral administration, alternatively by transdermal administration.

The dose of the pharmaceutical composition that is to be used depends on the particular condition being treated, the severity of the condition, the individual patient parameters including age, physical condition, size and weight, the duration of the treatment, the nature of concurrent therapy (if any), the specific route of administration and other similar factors that are within the knowledge and expertise of the health practioner. These factors are known to those of skill in the art and can be addressed with minimal routine experimentation.

### EXAMPLES

### Cell Fitness

The primary function of vascular SMCs in their quiescent state is to contract and provide vascular tone (Owens, 1995) (Sobue, 1998) (Rybalkin, 2003). The healthy SMC, which morphologically is similar to HITB5 or HITC6 SMCs that have been cultured in serum-free media for prolonged periods (more than 72 hours) is characterized by an elongated appearance, increased expression of SMC contractile proteins, such as h-caldesmon, metavinculin, smooth muscle myosin heavy chain and calponin and a marked decrease in apoptosis (Li, Circ. Res, 1999). We have found that HITB5 and HITC6 SMCs that are in this state have a significant increase in their transcript and protein levels of PBEF, demonstrating that PBEF is a novel factor involved in regulating SMC differentiation and maturation.

The Smooth Muscle Cell lines designated HITB5 and HITC6, in contrast to other vascular preparations, are capable of reversibly converting their phenotype between a spread, noncontractile state in the presence of serum and an elongated, contractile state after serum withdrawal (Li, Circ. Res, 1999). The withdrawal of serum induces in these cells a stress response, one that requires the cells to adapt to ensure survival. Primary cultures of SMCs cultured from explants are generally incapable of acquiring a truly, differentiated state in such stress conditions, and display an increased apoptotic rate. One of the hallmarks of HITB5 and HITC6 SMCs is their ability to adapt to media that is serum-free, suggesting that they possess the capability to activate a stress response that is sufficient to deal with the decreased abundance of nutrients and mitogens. Li, S., Sims, S., Jiao, Y., Chow, L. H., and Pickering, J. G. Evidence From a Novel Human Cell Clone That Adult Vascular Smooth Muscle Cells Can Convert Reversibly Between Noncontractile and Contractile Phenotypes. Circulation Research. 85(4): 338-348, 1999.

We show that vector-transduced or PBEF-overexpressing HITC6 SMCs in response to treatment with 5-phosphoribosyl-pyrophosphate (PRPP), demonstrating that the PRPP increases the health of cells compared to control cells;

The mechanism by which PBEF exerts its pleiotropic effects is controversial. It has been suggested that PBEF functions as a secreted cytokine (Samal, 1994) (Jia, 2004), while it has also been reported to be involved in the salvage pathway of NAD+ biosynthesis as a nicotinamide phosphoribosyltransferase (Rongvaux, 2002) (Martin, 2001). We believe that PBEF is an intracellular protein that catalyzes the rate-limiting reaction of the salvage pathway. This involves the conversion of nicotinamide (NAm) and 5-phosphoribosyl-pyrophosphate (PRPP) to nicontinamide mononucleotide (NMN) (Pilz, JBC, 1984) (Willis, Adv. Enzyme Reg., 1989). In resting human lymphocytes, the PRPP pools are very low (Snyder, JCI, 1976), and these cells were characterized by an impaired ability to generate ATP, inability to respond to mitogenic stimuli, while activation of these cells results in expansion of NAD+ and PRPP pools concomitant with the repair of DNA single-strand breaks (Johnstone, Eur. J. Biochem, 1984) (Berger, Exp. Cell Res., 1982) (Williams, Exp. Cell Res., 1985). Carson, D. A., Seto, S., and Wasson, D. B. Pyridine Nucleotide Cycling and Poly(ADP-Ribose) Synthesis in Resting Human Lymphocytes. Journal of Immunology. 138: 1904-1907, 1987. Willis, R. C., Nord, L. D., Fujitaki, J. M., and Robins, R. K. Potent and Specific Inhibitors of Mammalian Phosphoribosylpyrophosphate (PRPP) Synthetase. Advances in Enzyme Regulation. 28: 167-180, 1989.

PPRP and Nicotinamide are molecules critical for the regeneration of NAD in animal cells. NAD+ is a critical coenzyme involved in oxidation-reduction reactions in the cell. Cellular maintenance of NAD+ levels for redox reactions is likely tightly regulated due to its critical importance in energy generation. However, recent work has identified a number of other NAD+-dependent reactions that, unlike the redox system, deplete the free pool of intracellular NAD+. As such, regeneration of NAD+ can be achieved by de novo biosynthesis in primarily the liver or via the salvage pathway in the peripheral tissues (Magni, CMLS, 2004) (Bender, Brit. Jour. Nutr, 1988). The catabolism of NAD(P)+ in the liver results in the release of nicotinamide into the blood, which is utilized by peripheral tissues for the regeneration of NAD+ (Bender, 1988). Conversion of nicotinamide to nicotinic acid by nicotinamide deamidase is an important aspect of NAD+ generation in bacteria and yeast (Anderson, Nature, 2003) (Schuette, Am. J. Physiol, 1983). However, it is believed that nicotinamide deamidase is not expressed in mammals (Schuette, Am. J. Physiol, 1983) (Rongvaux, Bioessays, 2003). Therefore, conversion of nicotinamide and 5-phosphoribosyl pyrophosphate to NMN by PBEF is likely critical for the regeneration of NAD+ in peripheral tissues (Rongvaux, Eur. J. Biochem, 2002), such as SMCs. The marked upregulation of PBEF in differentiating SMCs demonstrating that the SMC differentiation is a NAD+ consuming process.

The present methods and formulations are adaptable in a myriad of ways to provide cyto-protection and healing to cells and tissues. As those of skill in the pharmaceutical or cosmetic arts will comprehend, numerous combinations and formulations of PBEF, PRPP and nicotinamide are within the scope and spirit of the invention, as are numerous variations of the present methods.

What is claimed is:

1. A method for treating diseases or conditions in an animal, said method comprising the step of optimizing the intracellular concentration of PBEF in the cells of at least one target tissue of said animal.

2. The method of claim 1, wherein said optimizing of said concentration of PBEF is effected by increasing the intracellular concentration of said PBEF of said animal a sufficient amount of PBEF.

3. The method of claim 1, wherein said optimizing of said concentration of PBEF is effected by administering to said animal a sufficient amount of PBEF to increase the intracellular concentration of said PBEF.

4. The method of claim 3, wherein said administering of PBEF is by at least one route, and said at least one route is one or more of injection, oral administration, anal or other colonic administration, inhalation, intra-peritoneal administration, topical administration, intra-organ administration, infusion of a target tissue, transdermal and parenteral administration, including intravenous, intraperitoneal, subcutaneous, intramuscular, trans-epithelial, nasal, intrapulmonary, intrathecal, rectal and topical modes of administration.

5. The method of claim 3, wherein said administering of PBEF is by at least one route, and said at least one route is one or more of the methods of gene therapy, including the use of one or more viral vectors.

6. The method of claim 3, wherein said one or more viral vectors are chosen from the group comprising adenoviruses, lentiviruses, adeno-associated viruses and non viral plasmid vectors.

7. The method of claim 3, wherein said increasing of said PBEF is effected by promoting the endogenous production of PBEF in the cells of at least one target tissue of said animal.

8. The method of claim 7, wherein said promotion of intracellular production of PBEF is effected by up-regulating the nucleic acid processes which support the production of PBEF.

9. The method of claim 7, wherein said promotion of intracellular production of PBEF is effected by up-regulating the nucleic acid processes which increase the endogenous production of PBEF.

10. The method of claim 7, wherein said promotion of intracellular production of PBEF is effected by down-regulating the nucleic acid processes which repress the production of PBEF.

11. The method of claim 1, wherein said optimization of PBEF is effected by increasing the intracellular concentration of at least one modulator of PBEF.

12. The method of claim 11, wherein said optimization of PBEF is effected by administering to said animal an effective amount of said modulator.

13. The method of claim 11, wherein said administering of said modulator is by at least one route, and said at least one route is one or more of injection, oral administration, anal or other colonic administration, inhalation, intra-peritoneal administration, topical administration, intra-organ administration, infusion of a target tissue, transdermal and parenteral administration, including intravenous, intraperitoneal, subcutaneous, intramuscular, trans-epithelial, nasal, intrapulmonary, intrathecal, rectal and topical modes of administration.

14. The method of claim 11, wherein said modulator is PRPP.

15. The method of claim 11, wherein said increase of PBEF is effected by promoting the endogenous production of PRPP in the cells of at least one target tissue of said animal.

16. The method of claim 14, wherein said promotion of intracellular production of PBEF is effected by up-regulating the nucleic acid processes which increase the production of PRPP.

17. The method of claim 14, wherein said promotion of intracellular production of PBEF is effected by down-regulating the nucleic acid processes which repress the production of PRPP.

18. The method of claim 14, wherein PRPP can be given in combination with at least one form of nicotinamide.

19. The method of claim 18, wherein said nicotinamide may be substituted or in the form of one or more of nicotinic acid; nicotinic acid ribonucleotide; nicotinic acid ribonucleotide, reduced form; nicotinamide ribonucleotide; nicotinamide ribonucleotide, reduced form; nicotinic acid adenine dinucleotide; nicotinic acid adenine dinucleotide, reduced form; nicotinamide adenine dinucleotide (NAD); nicotinamide adenine dinucleotide phosphate (NADP); nicotinamide adenine dinucleotide, reduced form (NADH); and nicotinamide adenine dinucleotide phosphate, reduced form (NADPH) and pharmaceutically acceptable salts thereof.

20. The method of claim 1, wherein said disease or condition is a vascular disease of one or more of the heart, blood vessels and other portions of the cardiovascular system

21. The method of claim 1, wherein said disease or condition is one or more of vascular insufficiency, vascular weakness, progeria, premature senescence of one or more tissues, aging, severe stress on one or more tissues, atherosclerosis, arteriolesclerosis and re-vascularization of injured or weakened tissues or organs.

22. The method of claim 1, wherein said severe stress on one or more tissue is due to one or more of injury, malnutrition, disease, toxic shock and exposure.

23. The method of claim 1, wherein said optimization of PBEF is effected by increasing the intracellular concentration of at least one precursor of PBEF.

24. The method of claim 23, wherein said increase of PBEF is effected by administering to said animal an effective amount of said precursor.

25. The method of claim 23, wherein said administering of said precursor is by at least one route, and said at least one route is one or more of injection, oral administration, anal or other colonic administration, inhalation, intra-peritoneal administration, topical administration, intra-organ administration, infusion of a target tissue, transdermal and parenteral administration, including intravenous, intraperitoneal, subcutaneous, intramuscular, trans-epithelial, nasal, intrapulmonary, intrathecal, rectal and topical modes of administration.

26. The method of claim 23, wherein said precursor is at least one form of nicotinamide.

27. The method of claim 26, wherein said nicotinamide may be substituted or in the form of one or more of nicotinic acid; nicotinic acid ribonucleotide; nicotinic acid ribonucleotide, reduced form; nicotinamide ribonucleotide; nicotinamide ribonucleotide, reduced form; nicotinic acid adenine dinucleotide; nicotinic acid adenine dinucleotide, reduced form; nicotinamide adenine dinucleotide (NAD); nicotinamide adenine dinucleotide phosphate (NADP); nicotinamide adenine dinucleotide, reduced form (NADH); and nicotinamide adenine dinucleotide phosphate, reduced form (NADPH) and pharmaceutically acceptable salts thereof.

28. The method of claim 1, wherein said animal is a human.

29. A composition for optimizing the intracellular concentration of NAD, said composition comprising an effective amount of PBEF.

30. The composition of claim 29, further comprising an effective amount of PRPP.

31. The composition of claim 30, further comprising an effective amount of nicotinamide.

32. The composition of claim 29, further comprising an effective amount of nicotinamide.

33. The composition of claim 32, wherein said nicotinamide may be substituted or in the form of one or more of nicotinic acid; nicotinic acid ribonucleotide; nicotinic acid ribonucleotide, reduced form; nicotinamide ribonucleotide; nicotinamide ribonucleotide, reduced form; nicotinic acid adenine dinucleotide; nicotinic acid adenine dinucleotide, reduced form; nicotinamide adenine dinucleotide (NAD); nicotinamide adenine dinucleotide phosphate (NADP); nicotinamide adenine dinucleotide, reduced form (NADH); and nicotinamide adenine dinucleotide phosphate, reduced form (NADPH) and pharmaceutically acceptable salts thereof.

34. The composition of claim 30, further comprising one or more of an effective amount of a pharmaceutically effective vehicle, a pharmaceutically effective diluent, a pharmaceutically effective cream, a pharmaceutically effective excipient, one or more pharmaceutically effective micelles, a pharmaceutically effective carrier, pharmaceutically acceptable concentrations of salt, buffering agents, preservatives and various compatible carriers.

35. The composition of claim 30, wherein said composition is adaptable for administration by at least one route, and said at least one route is one or more of injection, oral administration, anal or other colonic administration, inhalation, intra-peritoneal administration, topical administration, intra-organ administration, infusion of a target tissue, transdermal and parenteral administration, including intravenous, intraperitoneal, subcutaneous, intramuscular, trans-epithelial, nasal, intrapulmonary, intrathecal, rectal and topical modes of administration.

36. The composition of claim 29, wherein said composition is provided in the form of one or more of ingestible tablets, buccal tablets, troches, capsules, elixirs, suspensions, micelle encapsulations, syrups, wafers and the like, or enclosed or enclosable within hard or soft shell gelatin capsules.

37. The composition of claim 29, further comprising one or more of an effective amount of a cosmetically effective vehicle, a cosmetically effective diluent, a cosmetically effective cream, a cosmetically effective excipient, one or more cosmetically effective micelles, a cosmetically effective carrier, cosmetically acceptable concentrations of salt, buffering agents, preservatives and various cosmetically compatible carriers.

38. A composition for optimizing the intracellular concentration of NAD, said composition comprising an effective amount of PRPP.

39. The composition of claim 38, further comprising an effective amount of PBEF.

40. The composition of claim 39, further comprising an effective amount of nicotinamide.

41. The composition of claim 38, further comprising an effective amount of nicotinamide.

42. The composition of claim 41, wherein said nicotinamide may be substituted or in the form of one or more of nicotinic acid; nicotinic acid ribonucleotide; nicotinic acid ribonucleotide, reduced form; nicotinamide ribonucleotide; nicotinamide ribonucleotide, reduced form; nicotinic acid adenine dinucleotide; nicotinic acid adenine dinucleotide, reduced form; nicotinamide adenine dinucleotide (NAD); nicotinamide adenine dinucleotide phosphate (NADP); nicotinamide adenine dinucleotide, reduced form (NADH); and nicotinamide adenine dinucleotide phosphate, reduced form (NADPH) and pharmaceutically acceptable salts thereof.

43. The composition of claim 39, further comprising one or more of an effective amount of a pharmaceutically effective vehicle, a pharmaceutically effective diluent, a pharmaceutically effective cream, a pharmaceutically effective excipient, one or more pharmaceutically effective micelles, a pharmaceutically effective carrier, pharmaceutically acceptable concentrations of salt, buffering agents, preservatives and various compatible carriers.

44. The composition of claim 39, wherein said composition is adaptable for administration by at least one route, and said at least one route is one or more of injection, oral administration, anal or other colonic administration, inhalation, intra-peritoneal administration, topical administration, intra-organ administration, infusion of a target tissue, transdermal and parenteral administration, including intravenous, intraperitoneal, subcutaneous, intramuscular, trans-epithelial, nasal, intrapulmonary, intrathecal, rectal and topical modes of administration.

45. The composition of claim 38, wherein said composition is provided in the form of one or more of ingestible tablets, buccal tablets, troches, capsules, elixirs, suspensions, micelle encapsulations, syrups, wafers and the like, or enclosed or enclosable within hard or soft shell gelatin capsules.

46. The composition of claim 38, further comprising one or more of an effective amount of a cosmetically effective vehicle, a cosmetically effective diluent, a cosmetically effective cream, a cosmetically effective excipient, one or more cosmetically effective micelles, a cosmetically effective carrier, cosmetically acceptable concentrations of salt, buffering agents, preservatives and various cosmetically compatible carriers.
